(19) United States
(12) Patent Application Publication
(10) Pub. No.: US 2019/0298696 A1
(43) Pub. Date: Oct. 3, 2019

(54) DEUTERATED IMIDAZOLIDINEDIONE COMPOUNDS AND THEIR USES

(71) Applicant: Hinova Pharmaceuticals Inc., Chengdu (CN)

(72) Inventors: Xinghai Li, Torrance, CA (US); Yuanwei Chen, San Diego, CA (US)

(21) Appl. No.: 16/370,084

(22) Filed: Mar. 29, 2019

Related U.S. Application Data
(60) Provisional application No. 62/650,153, filed on Mar. 29, 2018.

(51) Int. Cl. A61K 31/4166; A61P 35/00

(52) U.S. Cl. CPC A61K 31/4166; A61K 9/0053; A61P 35/00

(57) ABSTRACT

Provided herein is a compound of Formula I having androgen receptor antagonistic properties and methods of using the compound to more efficiently and efficaciously reduce the concentration of prostate specific antigen (PSA) and treat male hormone-related diseases such as prostate cancer, breast cancer, alopecia, hair loss, acne and adolescent acne. Also provided are methods of reducing the plasma concentration of prostate specific antigen (PSA) in a subject with elevated PSA comprising administering to the subject a therapeutically effective amount of the compound.

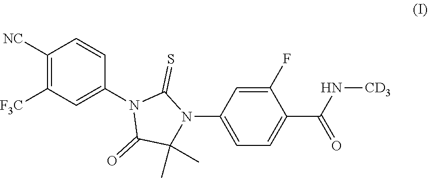 [SMILES: [2H]C([2H])([2H])NC(=O)c1ccc(N2C(=S)N(c3ccc(C#N)c(C(F)(F)F)c3)C(=O)C2(C)C)cc1F]

### BACKGROUND

Prostate cancer (prostatic carcinoma, abbreviated as PCa) is the most common malignant neoplasm in in male reproductive system. The incidence thereof increases with age, and differs significantly from region to region, which is higher in U.S. and Europe. Second to lung cancer, prostate cancer is the second cancer leading to death in men. Also, in the past, not much attention has been paid to prostate cancer in China, as it represented a small fraction of the diseases in the tumor spectrum. However, with the social development and progress in China, the aging of society, urbanization, westernization of dietary structure and advances in detection technology, the incidence of prostate cancer has significantly increased. A survey on prostate cancer which was completed by The Second Hospital of Tianjin Medical University and Diagnosis and Treatment of Prostate Cancer in Tianjin in 2011 showed that the incidence of prostate cancer in Tianjin was rapidly rising; the incidence of prostate cancer increased by 4 times in 20 years, and the number of patients with prostate cancer accounted for 13.4% of inpatient with urinary tract tumors. Prostate cancer which was a rare cancer in the past has become a common tumor in recent times, in many parts of the world.

An androgen receptor (AR) is a ligand-dependent trans-transcriptional regulatory protein with 110,000 dalton molecular weight. Androgen receptor plays a very important role in the pathogen and deterioration process of prostate cancer, and in male hormone-related diseases such as acne, male alopecia, and so on.

Traditional methods for treating prostate cancer include surgery or using androgen receptor antagonists such as bicalutamide (Casodex). However, patients tend to develop drug resistance after 2-4 years of treatment. Also, bicalutamide has side effects of stimulating the proliferation of cancer, and therefore patients must stop using bicalutamide after a period of time. Recent studies have found that bicalutamide will activate androgen receptors, thereby stimulating the proliferation of cancer.

Therefore, there is still a need in the art to develop compounds having superior pharmacodynamic properties for the treatment of prostate cancer.

### SUMMARY

Provided herein is a compound having androgen receptor antagonistic properties and methods of using the compound to more efficiently and efficaciously reduce the concentration of prostate specific antigen (PSA) and treat male hormone-related diseases such as prostate cancer.

Provided herein is an imidazolidinedione compound, 4-{3-[4-cyano-3-(trifluoromethyl)phenyl]-5,5-dimethyl-4-oxo-2-thio-1-imidazolidinyl}-2-fluoro-Ntrideuteromethyl benzamide of formula (I), referred to as HC-1119, or a crystal form, pharmaceutically acceptable salt, hydrate or solvate thereof:

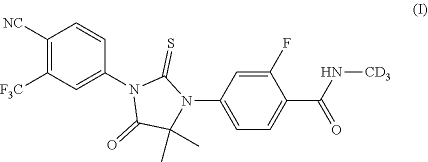 [SMILES: [2H]C([2H])([2H])NC(=O)c1ccc(N2C(=S)N(c3ccc(C#N)c(C(F)(F)F)c3)C(=O)C2(C)C)cc1F]

as well as pharmaceutical compositions thereof.

Also provided herein is a method of reducing the plasma concentration of prostate specific antigen (PSA) in a subject with elevated PSA comprising administering a therapeutically effective amount of HC-1119 or a crystal form, pharmaceutically acceptable salt, hydrate or solvate thereof or a pharmaceutical composition comprising HC-1119 to the subject.

Also provided herein is a method of treating a male hormone-related disease in a subject with elevated PSA comprising administering HC-1119, or a crystal form, pharmaceutically acceptable salt, hydrate or solvate thereof or a pharmaceutical composition comprising HC-1119 to the subject.

Also provided herein is a method of producing plasma concentrations of a combination of HC-1119 and its second metabolic product (HC-1119-M2) in a subject that is at least 50% more than a combination of enzalutamide and its second metabolic product (M2) produced with an identical dosage of enzalutamide by administering at least 40 mg HC-1119.

Also provided herein is a method of producing plasma concentrations of a combination of HC-1119 and its second metabolic product (HC-1119-M2) in a subject that is at least 10 μg/mL at steady state in a patient by administering at least 40 mg HC-1119.

Also provided herein is a method of reducing inter-patient variability in the plasma concentrations or AUC of a nonsteroidal antiandrogen (NSAA) compound comprising administering to a subject in need thereof, a therapeutically effective amount of a deuterated analogue of the NSAA compound. In one embodiment, the inter-patient variability in Cₘₐₓ, C₂₄ₕ, and/or C_{trough} are reduced.

Also provided herein is a method of reducing the side effects of a NSAA compound comprising administering to a subject in need thereof, a therapeutically effective amount of a deuterated analogue of the NSAA compound.

### BRIEF DESCRIPTION OF FIGURES

FIGS. 1A and 1B of FIG. 1 show the results of the HC-1119 Phase 1a clinical studies.

FIGS. 2A and 2B show the results of the Phase I/II studies with the similar drug, enzalutamide.

FIG. 3 provides the clinical PK parameters (40 mg QD) from the HC-1119 trial.

FIG. 4 provides the clinical PK parameters (80 mg QD) from the HC-1119 trial.

FIG. 5 provides the clinical PK parameters (160 mg QD) from the HC-1119 trial.

FIG. 6 provides the clinical PK parameters (200 mg QD) from the HC-1119 trial.

FIG. 7 provides the clinical PK parameters from an enzalutamide study.

FIG. 8 provides some PK parameters for 80 mg HC-1119 in comparison with those of 160 mg of enzalutamide.

FIG. 9 shows the plasma concentration versus time curve at week 12 for patients dosed with 40 mg HC-1119.

FIG. 10 shows the plasma concentration versus time curve at week 12 for patients dosed with 80 mg HC-1119.

FIG. 11 shows the plasma concentration versus time curve at week 12 for patients dosed with 160 mg HC-1119.

FIG. 12 shows the plasma concentration versus time curve at week 12 for patients dosed with 200 mg HC-1119.

FIG. 13 shows the M0 C₂₄ₕ changes during the 12 weeks of treatment for patients dosed with 40 mg, 80 mg, 160 mg and 200 mg of HC-1119.

FIG. 14 shows the M1 C₂₄ₕ changes during the 12 weeks of treatment for patients dosed with 40 mg, 80 mg, 160 mg and 200 mg of HC-1119.

FIG. 15 shows the M2 C₂₄ₕ changes during the 12 weeks of treatment for patients dosed with 40 mg, 80 mg, 160 mg and 200 mg of HC-1119.

FIGS. 16A and 16B show the (M0+M2) changes in C₂₄ₕ at steady state for patients dosed with either 80 mg HC-1119 (FIG. 16A) or 160 mg enzalutamide (FIG. 16B).

### DETAILED DESCRIPTION

The invention is based on the discovery that the deuterated imidazolidinedione compound of formula (I) of the present invention, or a crystal form, pharmaceutically acceptable salt, hydrate or solvate thereof have excellent pharmacokinetics and/or pharmacodynamic properties, and is, therefore, more suitably used as androgen receptor antagonists, and in the preparation of drugs/medicaments for treating androgen-related diseases. The invention provides an imidazolidinedione compound, 4-{3-[4-cyano-3-(trifluoromethyl)phenyl]-5,5-dimethyl-4-oxo-2-thio-1-imidazolidinyl}-2-fluoro-Ntrideuteromethyl benzamide, of formula (I) above, or a crystal form, pharmaceutically acceptable salt, hydrate or solvate thereof.

The compound of the invention possesses outstanding androgen receptor (AR) antagonism, therefore, the compound of the invention and the crystal forms, the pharmaceutically acceptable inorganic or organic salts, hydrates or solvates thereof, and pharmaceutical compositions comprising the compound of the invention as the main active ingredient, can be used for treating, preventing and alleviating diseases mediated by the AR.

The terms “compound of the invention,” the “compound of formula (I)” and “HC-1119” are used interchangeably herein and all refer to 4-{3-[4-cyano-3-(trifluoromethyl)phenyl]-5,5-dimethyl-4-oxo-2-thio-1-imidazolidinyl}-2-fluoro-N-trideuteromethyl benzamide.

A pharmaceutical composition of the invention comprises a safe and therapeutically effective amount of the compound of the invention or the pharmaceutical acceptable salts thereof and pharmaceutically acceptable excipients or carriers. As used herein, a “therapeutically effective amount” refers to an amount of the compounds which is sufficient to improve the patient's condition and would not induce serious side effect.

As used herein, a “pharmaceutically acceptable carrier” refers to one or more compatible solid or liquid fillers or gel material, which are suitable for human, and must have sufficient purity and sufficiently low toxicity. “Compatibility” herein means that the components of the compositions can be blended with the compounds of the invention or with each other, and would not significantly reduce the efficacy of the compounds. Some examples of pharmaceutically acceptable carriers include cellulose and the derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agents, flavoring agents, stabilizers, antioxidants, preservatives, pyrogen-free water, etc.

As used herein, a cancer “determined to be drug resistant,” as used herein, refers to a cancer that is drug resistant, based on unresponsiveness or decreased responsiveness to a chemotherapeutic agent, or is predicted to be drug resistant based on a prognostic assay (e.g., a gene expression assay).

As used herein, a subject with an “elevated PSA concentration” or “elevated PSA level” has a PSA concentration that is about 5 ng/ml or higher. In some embodiments, The PSA concentration in a subject with an elevated PSA concentration is about 6 ng/ml or higher or about 7 ng/ml or higher or about 8 ng/ml or higher or about 9 ng/ml or higher. In one embodiment, the PSA concentration in a subject with an elevated PSA concentration is about 10 ng/ml or higher.

The terms “subject” or “patient” are used interchangeably herein to mean all mammals including humans. Examples of subjects include humans, cows, dogs, cats, horses, goats, sheep, pigs, and rabbits. In one embodiment, the patient is a human.

As used herein, “unit dosage form” refers to a physically discrete unit suitable as a unitary dosage for a human or an animal. Each unit dosage form can contain a predetermined amount of a therapeutic agent calculated to produce a desired effect.

In one aspect, a unit dosage form of the present invention contains the compound of formula I, or a crystal form, pharmaceutically acceptable salt, hydrate or solvate thereof, and at least a pharmaceutically acceptable carrier. In some embodiments, the C_{trough} ratio at steady state between the compound of Formula I and M2 ranges from about 4.5:1 to about 5.5:1, from about 4.8:1 to about 5.5:1, from about 5:1 to about 5.5:1, or from about 5:1 to about 6:1, when the unit dosage form is administered once a day, twice a day, or once every N days (N=2 to 8, inclusive) to a subject in need thereof. In some embodiments, the unit dosage form contains the compound of formula I in an amount of at least 10 mg, at least 40 mg, at least 80 mg, at least 100 mg, at least 160 mg, or at least 200 mg, or a crystal form, pharmaceutically acceptable salt, hydrate or solvate derived or prepared from such amount of the compound of Formula I. In some embodiments, the unit dosage form contains the compound of formula I in an amount of 10 mg, 20 mg, 30 mg, 40 mg, 50 mg, 60 mg, 70 mg, 80 mg, 90 mg, 100 mg, 120 mg, 140 mg, 160 mg, 200 mg, or 300 mg, or a crystal form, pharmaceutically acceptable salt, hydrate or solvate derived or prepared from such amount of the compound of Formula I.

It will also be appreciated that the compound, composition and unit dosage form of the present invention can be formulated and employed in combination therapies, that is, the compounds, pharmaceutical compositions or unit dosage form can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

A related aspect provides a kit which includes the unit dosage form of the present invention and at least one additional agent. Examples of the additional agent includes PD1 inhibitors, PD-L1 inhibitors, and PARP inhibitors. Examples of the PD1 inhibitors and PD-L1 inhibitors include nivolumab (i.e., MDX-1 106, BMS-936558, ONO-4538); CT-011; AMP-224; pembrolizumab; pidilizumab; or MK-3475); BMS936559 (i.e., MDX-1 105); MEDI4736; MSB001071 8C (avelumab); MPDL-3280A); SHR-1210, IBI308, BGB-A317; JS001, GLS-010, GB226, HLX10, AK103, AK104, AK105, AK112, SSI-361, JY034, KN035, SHR1316, TQB2450, KL-A167, CS1001, STI-A1014, JS003, AK106, HLX-09, atezolizumab and MEDI4736. Examples of the PARP inhibitors include olaparib, Rucaparib, Niraparib, talazoparib, pamiparip, Zejula, Veliparib, CEP-8983, BGP-15, JPI-289, Fluzoparib, Fuzuopali, BGB-290, ABT-767, MP-124, Simmiparib, Meifupairui, IMP-4297, and WXFL10040340.

In one aspect, the invention provides a method of reducing the plasma concentration of prostate specific antigen (PSA) in a subject with elevated PSA comprising administering a therapeutically effective amount of the compound of Formula I, or a crystal form, pharmaceutically acceptable salt, hydrate or solvate thereof or a pharmaceutical composition comprising a therapeutically effective amount of the compound of Formula I to the subject.

In one embodiment, the plasma concentration of PSA in a subject with elevated PSA is lowered by at least 50%. In another embodiment, the plasma concentration of PSA in a subject with elevated PSA is lowered by at least 55%. In another embodiment, the plasma concentration of PSA in a subject with elevated PSA is lowered by at least 60%. In another embodiment, the plasma concentration of PSA in a subject with elevated PSA is lowered by at least 65%. In another embodiment, the plasma concentration of PSA in a subject with elevated PSA is lowered by at least 70%. In another embodiment, the plasma concentration of PSA in a subject with elevated PSA is lowered by at least 75%. In another embodiment, the plasma concentration of PSA in a subject with elevated PSA is lowered by at least 80%. In another embodiment, the plasma concentration of PSA in a subject with elevated PSA is lowered by at least 85%.

In another embodiment, the plasma concentration of PSA in a subject with elevated PSA is lowered by over 89%. In yet another embodiment, the plasma concentration of PSA in a subject with elevated PSA is lowered by at least 90%. In some embodiments, the plasma concentration of PSA in a subject with elevated PSA is lowered by at least 91% or at least 92% or at least 93% or at least 94%. In some embodiments, the plasma concentration of PSA in a subject with elevated PSA is lowered by at least 95% or at least 96% or at least 97% or at least 97.5%. In some embodiments, the plasma concentration of PSA in a subject with elevated PSA is lowered by at least 98% or at least 98.5% or at least 99% or at least 99.5%. In some embodiments, the plasma concentration of PSA in a subject with elevated PSA is lowered by at least 99.6% or at least 99.7% or at least 99.8% or at least 99.9%.

In one embodiment, the plasma concentration of PSA in a subject with elevated PSA is lowered by at least 50% over a period of five (5) weeks or more. In another embodiment, the plasma concentration of PSA in a subject with elevated PSA is lowered by at least 50% over a period of six (6) weeks or more. In yet another embodiment, the plasma concentration of PSA in a subject with elevated PSA is lowered by at least 50% over a period of eight (8) weeks or more. In yet another embodiment, the plasma concentration of PSA in a subject with elevated PSA is lowered by at least 50% over a period of ten (10) weeks or more. In yet another embodiment, the plasma concentration of PSA in a subject with elevated PSA is lowered by at least 50% over a period of twelve (12) weeks or more.

Prostate-specific antigen (PSA) is a protein produced by the prostate gland. Elevated PSA levels may indicate prostate cancer or a noncancerous condition such as prostatitis, or an enlarged prostate. Most men have PSA levels under 4 ng/mL and this has traditionally been used as the cutoff for concern about the risk of prostate cancer.

For this reason, measurement of PSA in the blood has been used as a screening test for prostate cancer. However, the PSA test was first developed only to monitor men who had a history of prostate cancer. No single normal level has been established. Historically, a level of 4.0 ng/mL or higher was used to justify a biopsy of the prostate (a sample of prostate tissue) to try and determine if a man has prostate cancer. However, this practice has been changing and other factors are being considered in the decision to perform a prostate biopsy.

In one embodiment, the subject is a human suffering from an androgen-receptor-activity related disease. The androgen-receptor-activity-related disease could be any male hormone related or male hormone-associated disease, including, but not limited to, prostate cancer, breast cancer, acne, pimples, male alopecia, hair regeneration, and so on.

In one embodiment, the method further includes, before the administration step, determining or diagnosing the subject as currently having, or having been previously diagnosed with prostate cancer. In one embodiment, the androgen-receptor-activity-related disease is prostate cancer. In one embodiment, the subject either currently has, or has previously been diagnosed with prostate cancer. The prostate cancer could be any type of prostate cancers. The prostate cancer could be any type of AR positive prostate cancers, including, but not limited to, castration resistant prostate cancer (CRPC) or metastatic castration resistant prostate cancer (mCRPC) or non-metastatic castration resistant prostate cancer (nmCRPC).

The subject could be one who is new to any form of treatment for prostate cancer, or one who has previously been treated for prostate cancer. In one embodiment, the subject is one who has never been treated for prostate cancer. In another embodiment, the subject was previously diagnosed with prostate cancer and has undergone previous treatment for prostate cancer. In one embodiment, the prostate cancer is any wild type AR positive prostate cancer and AR mutant positive prostate cancer. In one embodiment, the prostate cancer is castration resistant prostate cancer (CRPC). In one embodiment, the prostate cancer is metastatic castration resistant prostate cancer (mCRPC). In one embodiment, the prostate cancer is non-metastatic castration resistant prostate cancer (nmCRPC).

In one embodiment, the method further comprises before the administration step, determining the prostate cancer in the subject as being resistant to the treatment of a single androgen receptor antagonist selected from the group consisting of Flutamide, Nilutamide, Bicalutamide, Abiraterone Acetate, Apalutamide, Darolutamide, Proxalutamide, TRC-253, ONC1-13B, TAK-683, TAK-448, Enzalutamide, SHR3680, GT0918 (Proxalutamide), and ARN509.

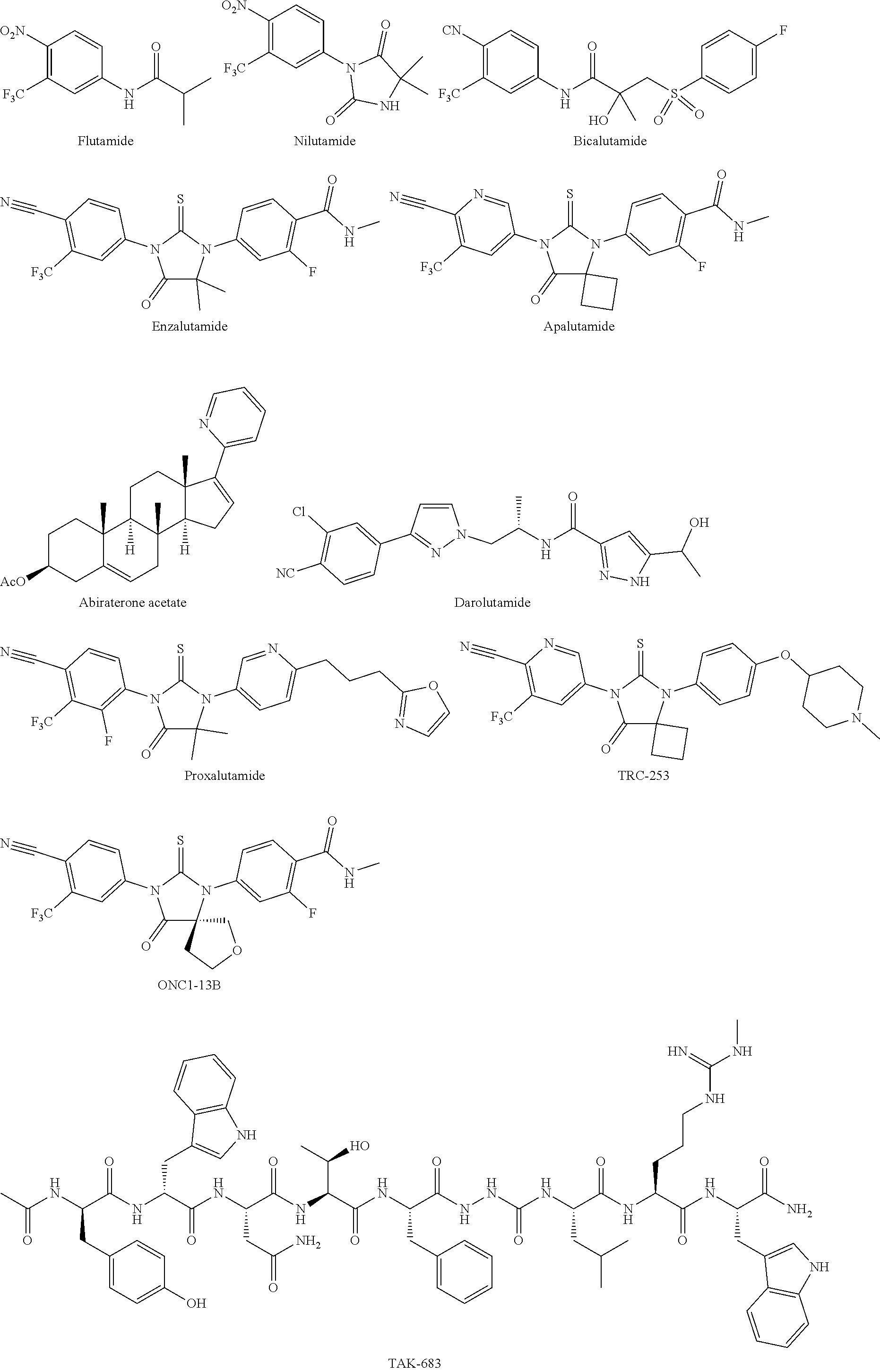

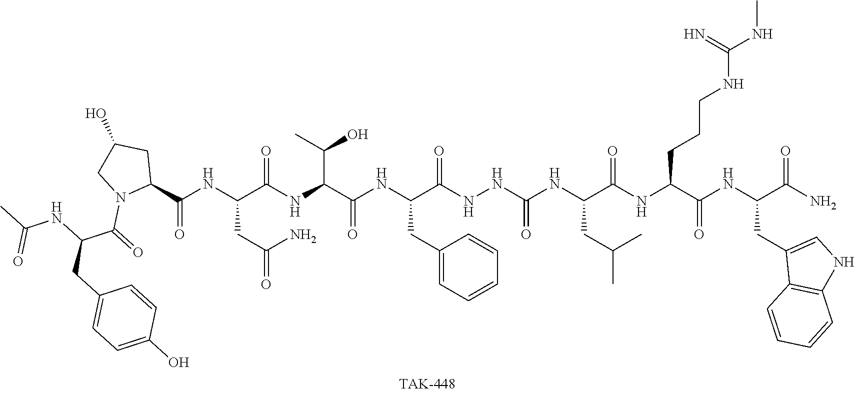 [SMILES: CNC(=N)NCCC[C@H](NC(=O)[C@H](CC(C)C)NC(=O)NNC(=O)[C@H](Cc1ccccc1)NC(=O)[C@@H](NC(=O)[C@H](CC(N)=O)NC(=O)[C@@H]1C[C@@H](O)CN1C(=O)[C@@H](Cc1ccc(O)cc1)NC(C)=O)[C@@H](C)O)C(=O)N[C@@H](Cc1c[nH]c2ccccc12)C(N)=O]

The compound of Formula I (HC-1119) or a crystal form, pharmaceutically acceptable salt, hydrate or solvate thereof or a pharmaceutical composition comprising the compound of Formula I is administered to subjects in need thereof in a pharmaceutically or therapeutically effective amount. For a person weighing about 60 kg, the dose is usually about 1 mg to about 2000 mg, preferably about 10 mg to about 500 mg. In one embodiment, the dose or unit dose of the compound of Formula I is about 20 mg to about 250 mg. In another embodiment, the dose or unit dose is about 30 mg to about 200 mg. In yet another embodiment, the dose or unit dose is about 10 mg of the compound of Formula I. In yet another embodiment, the dose or unit dose is about 20 mg of the compound of Formula I. In yet another embodiment, the dose or unit dose is about 30 mg of the compound of Formula I. In yet another 15 embodiment, the dose or unit dose is about 40 mg of the compound of Formula I. In yet another embodiment, the dose or unit dose is about 60 mg of the compound of Formula I. In yet another embodiment, the dose or unit dose is about 80 mg of the compound of Formula I. In yet another embodiment, the dose or unit dose is about 100 mg of the compound of Formula I. In yet another embodiment, the dose or unit dose is about 120 mg of the compound of Formula I. In yet another embodiment, the dose or unit dose is about 160 mg of the compound of Formula I. In yet another embodiment, the dose or unit dose is about 200 mg of the compound of Formula I. In any of the embodiments described herein, the dose or unit dose may contain a crystal form, pharmaceutically acceptable salt, hydrate or solvate or a pharmaceutical composition derived or prepared from the above described amount of the compound of Formula I.

In one embodiment, the C_{trough} ratio at steady state between the compound of formula I and its metabolite I-M2 ranges from about 5:1 to about 6:1, from about 5:1 to about 5.5:1, or from about 5.5:1 to about 6:1 wherein I-M2 is represented as

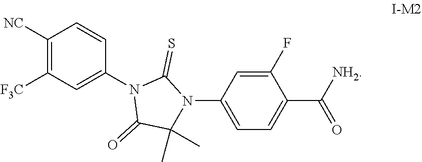 [SMILES: CC1(C)C(=O)N(c2ccc(C#N)c(C(F)(F)F)c2)C(=S)N1c1ccc(C(N)=O)c(F)c1]

In one embodiment, provided herein is a method of reducing the plasma concentration of prostate specific antigen (PSA) in a subject with elevated PSA by at least 50% comprising administering to said subject at least 10 mg of the compound of Formula I. In another embodiment, provided herein is a method of reducing the plasma concentration of prostate specific antigen (PSA) in a subject with elevated PSA by at least 50% comprising administering to said subject at least 20 mg of the compound of Formula I. In yet another embodiment, provided herein is a method of reducing the plasma concentration of prostate specific antigen (PSA) in a subject with elevated PSA by at least 50% comprising administering to said subject at least 40 mg of the compound of Formula I. In some embodiments, provided herein is a method of reducing the plasma concentration of prostate specific antigen (PSA) in a subject with elevated PSA by at least 50% comprising administering to said subject at least 80 mg, or at least 100 mg, or at least 120 mg, or at least 160 mg, or at least 200 mg of the compound of Formula I, or a crystal form, pharmaceutically acceptable salt, hydrate or solvate or a pharmaceutical composition derived or prepared from the above described amount of the compound of Formula I.

Of course, the particular dose would depend on several factors, including, but not limited to, level of PSA, the subject's age, weight, health status and responsiveness to the treatment as well as the frequency and route of administration, which are within the scope of one of skill in the art.

In one embodiment, the dosage is kept consistent throughout the course of treatment. In another embodiment, the dosage is changed during the course of treatment. In one embodiment, the first dose is higher than subsequent doses. In another embodiment, the first dose is lower than subsequent doses. In yet another embodiment, the dose varies each time depending on the subject's need as determined based on the factors provided above as well as other relevant factors as determined by one of skill in the art.

The frequency with which the unit dosage form the compound of Formula I or a crystal form, pharmaceutically acceptable salt, hydrate or solvate thereof or a pharmaceutical composition comprising the compound of Formula I, is administered to subjects in need thereof will vary according to dosage, the subject's health status and responsiveness to the treatment, the route of administration as well as other relevant factors as determined by one of skill in the art. In one embodiment, the unit dosage form is administered to a subject in need thereof one or more times a day. In one embodiment, the unit dosage form is administered to a subject in need thereof once a day. In another embodiment, HC-1119 is administered to a subject in need thereof twice a day. In another embodiment, the unit dosage form is administered to a subject in need there of once every N days, where N is an integer between two (2) and eight (8), both inclusive.

The compound of this invention, or a crystal form, pharmaceutically acceptable salt, hydrate or solvate thereof or a pharmaceutical composition comprising the compound, may be administered by any number of routes including, but not limited to, oral, parenteral, intravenous, intramuscular, intra-arterial/intraarticular, intramedullary, intraperitoneal, intrathecal, intraventricular, transdermal, transcutaneous, topical, subcutaneous, intranasal, transmucosal, enteral, dermal, buccal, sublingual, intraocular, intravaginal or rectal routes. Hyposprays may also be used to administer the pharmaceutical compositions of the invention. Typically, the therapeutic composition may be prepared as injectables, either as liquid solutions or suspensions. Solid forms suitable for solution in, or suspension in, liquid vehicles prior to injection may also be prepared. In one embodiment, the therapeutic compound or composition is administered orally. In another embodiment, the therapeutic compound or composition is administered as an injection, or as a capsule, tablet, pill, powder or as granules.

Solid dosage forms for oral administration include capsules, tablets, pills, powders and granules. In these solid dosage forms, the active compounds are mixed with at least one conventional inert excipient (or carrier), such as sodium citrate or dicalcium phosphate, or mixed with the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and gum arabic; (c) humectant, such as, glycerol; (d) disintegrating agents such as agar, calcium carbonate, potato starch or tapioca starch, alginic acid, certain complex silicates, and sodium carbonate; (e) dissolution-retarding agents, such as paraffin; (f) absorption accelerators, for example, quaternary ammonium compounds; (g) wetting agents, such as cetyl alcohol and single glyceryl stearate; (h) adsorbents, for example, kaolin; and (i) lubricants such as talc, stearin calcium, magnesium stearate, solid polyethylene glycol, sodium lauryl sulfate, or the mixtures thereof. In capsules, tablets and pills, the dosage forms may also contain buffer.

The solid dosage forms such as tablets, sugar pills, capsules, pills and granules can be prepared by using coating and shell material, such as enteric coatings and other materials known in the art. They can contain opaque agent, and the release of the active compounds or compounds in such compositions can be delayed for releasing in certain portion of the digestive tract. Instance of the embedding components can be polymers and waxes. If necessary, the active compounds and one or more above excipients can be prepared into microcapsules.

Liquid dosage forms for oral administration include pharmaceutically acceptable emulsions, solutions, suspensions, syrups or tinctures. In addition to the active compounds, the liquid dosage forms may contain conventional inert diluent known in the art, such as water or other solvent, solubilizer and emulsifier, for example, ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the mixtures thereof and so on.

Besides the inert diluents, the composition may also contain additives such as wetting agents, emulsifiers, and suspending agent, sweetener, flavoring agents and perfume.

In addition to the active compounds, the suspension may contain suspending agent, for example, ethoxylated isooctadecanol, polyoxyethylene sorbitol and sorbitan esters, microcrystalline cellulose, methanol aluminum and agar, or the mixtures thereof and so on.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and the suitable mixtures thereof.

The dosage forms of the compound of the invention for topical administration include ointments, powders, patches, aerosol, and inhalants. The active ingredients are mixed with physiologically acceptable carriers and any preservatives, buffers, or propellant if necessary, under sterile conditions.

The compound of the invention can be administered alone, or in combination with other pharmaceutically acceptable compounds.

In another aspect, the invention provides a method of treating a male hormone-related disease or condition in a subject with elevated PSA comprising administering the compound of Formula I, or a crystal form, pharmaceutically acceptable salt, hydrate or solvate thereof or a pharmaceutical composition comprising the compound of Formula I to the subject.

As described above, the male hormone-related disease could be any disease or condition, now known, or later discovered, that is associated with activity of the androgen receptor. In one embodiment, the disease or condition is prostate cancer, breast cancer, acne, pimples, male alopecia, hair regeneration, etcetera. The subject could be one who has been previously diagnosed or is currently diagnosed with the disease or condition. The subject may, but need not, have been previously treated for the disease or condition.

In another aspect, the invention provides a method of producing plasma concentrations of a combination of the compound of Formula I and its second metabolic product (I-M2 or HC-1119-M2) in a subject that is at least 50% more than a combination of the enzalutamide and its second metabolic product (M2) produced with an identical dosage of enzalutamide.

The structural formula for HC-1119, 4-{3-[4-cyano-3-(trifluoromethyl)phenyl]-5,5-dimethyl-4-oxo-2-thio-1-imidazolidinyl}-2-fluoro-N-trideuteromethyl benzamide, is provided above as formula (I). As would be known to one of skill in the art, enzalutamide has the same structural formula except that in the case of enzalutamide the hydrogens on the amide methyl (—CH₃) group remain hydrogens and are not substituted with deuterium. These compounds, enzalutamide and the compound of Formula I are also referred to herein as M0 and HC-1119-M0 or I-M0, respectively.

Provided below are the first metabolic product (M1) and second metabolic product (M2), respectively, of enzalutamide and the compound of Formula I. Based on in vitro experiments, M1 is not active, whereas M2 exhibited similar in vitro activity to enzalutamide. While it is noted (based on the structures below) that deuteration (at the now eliminated methyl group) is irrelevant in the case of M1 and M2, just in order to distinguish the source of the metabolic product, the first and second metabolic product of the compound of Formula I (HC-1119) will be referred to as I-M1 (HC-1119-M1) and I-M2 (HC-1119-M2), respectively, whereas the first and second metabolic product of enzalutamide will be referred to as M1 and M2, respectively. M1 is identical to I-M1 (HC-1119-M1) and M2 is identical to I-M2 (HC-1119-M2).

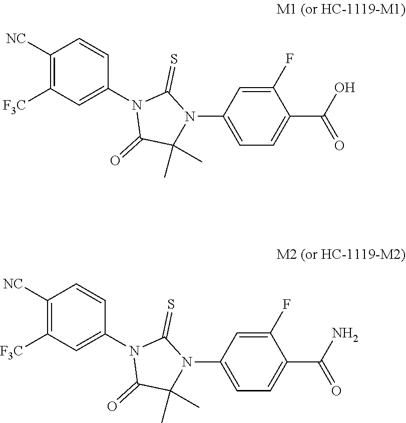 [SMILES: CC1(C)C(=O)N(c2ccc(C#N)c(C(F)(F)F)c2)C(=S)N1c1ccc(C(=O)O)c(F)c1 ; CC1(C)C(=O)N(c2ccc(C#N)c(C(F)(F)F)c2)C(=S)N1c1ccc(C(N)=O)c(F)c1]

In one embodiment, the invention provides a method of producing plasma levels of the combination of HC-1119-M0 (HC-1119) and HC-1119-M2 (the second metabolic product of HC-1119) of about 10 μg/ml at steady state. In another embodiment, the plasma levels of the combination of HC-1119-M0 and HC-1119-M2 are >10 μg/ml. In yet another embodiment, the plasma levels of the combination of HC-1119-M0 and HC-1119-M2 are about 20 μg/ml. In yet another embodiment, the plasma levels of the combination of HC-1119-M0 and HC-1119-M2 are about 30 μg/ml. In yet another embodiment, the plasma levels of the combination of HC-1119-M0 and HC-1119-M2 are about 40 μg/ml. In yet another embodiment, the plasma levels of the combination of HC-1119-M0 and HC-1119-M2 are about 50 μg/ml. In yet another embodiment, the plasma levels of the combination of HC-1119-M0 and HC-1119-M2 are >50 μg/ml.

The above mentioned plasma levels of the combination of HC-1119-M0 and HC-1119-M2 can be achieved by administrating to the subject a unit dosage form containing a suitable amount of the compound of Formula I (HC-1119) in a desirable frequency or interval. As stated above, the compound of Formula I may be administered to a subject in need thereof once a day, or twice a day, or once in two (2) days, or once in three (3) days, or once a week. It will be appreciated that if a higher dosage of the compound of Formula I is administered, the frequency of dosing will be less to achieve the same levels. In one embodiment, at least 10 mg of the compound of Formula I is administered to the subject. In another embodiment, at least 20 mg of the compound of Formula I is administered to the subject. In yet another embodiment, at least 30 mg of the compound of Formula I is administered to the subject. In yet another embodiment, at least 40 mg of the compound of Formula I is administered to the subject. In yet another embodiment, at least 60 mg of the compound of Formula I is administered to the subject. In yet another embodiment, 80 mg of the compound of Formula I is administered to the subject. In yet another embodiment, at least 100 mg of the compound of Formula I is administered to the subject. In yet another embodiment, at least 120 mg of the compound of Formula I is administered to the subject. In yet another embodiment, 160 mg of the compound of Formula I is administered to the subject. In yet another embodiment, 200 mg of the compound of Formula I is administered to the subject. The administered unit dosage form may also contain a crystal form, pharmaceutically acceptable salt, hydrate or solvate or a pharmaceutical composition derived or prepared from the above described amount of the compound of Formula.

In one embodiment, the invention provides a method of producing plasma concentrations of a combination of the compound of Formula I and I-M2 (HC-1119-M2) in a subject that is at least about 10 μg/mL at steady state by administering at least 40 mg the compound of Formula I.

In another embodiment, the invention provides a method of producing plasma concentrations of a combination of HC-1119 and HC-1119-M2 in a subject that is at least about 20 μg/mL at steady state by administering at least 80 mg HC-1119.

In yet another embodiment, the invention provides a method of producing plasma concentrations of a combination of HC-1119 and HC-1119-M2 in a subject that is at least about 30 μg/mL at steady state by administering at least 160 mg HC-1119.

In another embodiment, the invention provides a method of producing c_{trough} at steady state of more than about 10, more than about 12, more than about 16, more than about 18, more than about 20, or more than about 22 ug/ml in a subject comprising administering to the subject about 40 mg, about 80 mg or about 100 mg of the compound of Formula I.

In yet another aspect, the invention provides a method of reducing inter-patient variability in the plasma concentrations and/or AUC of a nonsteroidal antiandrogen (NSAA) compound comprising administering to a subject in need thereof, a unit dosage form containing a therapeutically effective amount of a deuterated analogue of the NSAA compound. The reduced inter-patient variability may, in one embodiment, be demonstrated by, for example, a smaller percent coefficient of variation (CV %) of the standard deviation (SD) comparing to that of enzalutamide. In one embodiment, the inter-patient variability in Cₘₐₓ, C₂₄ₕ, and/or C_{trough} are reduced.

The invention also provides a method of reducing the side effects of a NSAA compound (e.g. Enzalutamide) comprising administering to a subject in need thereof, a therapeutically effective amount of a deuterated analogue of the NSAA compound. Side effect from administration of Enzalutamide includes back pain, joint aches, musculoskeletal pain, diarrhea, hot flashes, peripheral edema (swelling in your hand, arms, legs, or feet), low white blood cell count, headache, upper respiratory tract infection, dizziness, and muscle weakness.

A nonsteroidal antiandrogen (NSAA) compound is an antiandrogen compound with a nonsteroidal chemical structure. They are typically selective and full or silent antagonists of the androgen receptor (AR) and act by directly blocking the effects of androgens like testosterone and dihydrotestosterone. NSAAs are used in the treatment of androgen-dependent conditions in men and women. Unlike steroidal antiandrogen compounds, NSAAs have little or no capacity to activate the AR, show no off-target hormonal activity such as progestogenic, glucocorticoid, or antimineralocorticoid activity, and lack antigonadotropic effects. For these reasons, they have improved efficacy and selectivity as antiandrogens and do not lower androgen levels. Instead, they act solely by directly blocking the actions of androgens at the level of their biological target, the AR.

Inter-patient variability in the plasma concentrations (for example, Cₘₐₓ, C₂₄ₕ, or C_{trough}) or AUC of any NSAA compound, now known, or later developed, can be reduced by administering to a patient in need thereof, a therapeutically effective amount of a deuterated analogue of the NSAA compound.

As described above, the subject or patient may have any disease or condition that is male hormone-related that is now known, or later discovered, and that is associated with activity of the androgen receptor. In one embodiment, disease or condition is prostate cancer, breast cancer, acne, pimples, male alopecia, hair regeneration, etcetera. The subject could be one who has been previously diagnosed or is currently diagnosed with the disease or condition. The subject may, but need not, have been previously treated for the disease or condition.

In one embodiment, the subject is one who was previously diagnosed with prostate cancer and has undergone previous treatment for prostate cancer. The subject could be who was diagnosed with any type of prostate cancer, including, but not limited to, CRPC and mCRPC and nmCRPC, and has undergone previous treatment for the same. In another embodiment, the subject is one who has never been treated for prostate cancer.

In one embodiment, the efficacy of the NSAA compound is increased due to the reduced inter-patient variability. In another embodiment, the plasma concentration of the NSAA compound is increased due to the reduced inter-patient variability. In yet another embodiment, the T_{1/2} for the NSAA compound is increased due to the reduced inter-patient variability. In yet another embodiment, the incidents or severity of adverse effects of the NSAA compound is decreased due to the reduced inter-patient variability.

In one embodiment, the NSAA compound is enzalutamide and the deuterated analogue of the NSAA compound is the compound of Formula I (HC-1119). As described above, several combinations of dosage and frequency of administration can be used to achieve the desired effect. The administered unit dosage form contains a predetermined amount or dose of HC-1119, or a crystal form, pharmaceutically acceptable salt, hydrate or solvate thereof calculated to produce a desired effect. In one embodiment, the unit dose is about 10 mg of HC-1119. In another embodiment, the unit dose is about 20 mg of HC-1119. In yet another embodiment, the dose is about 30 mg of HC-1119. In yet another embodiment, the unit dose is about 40 mg of HC-1119. In yet another embodiment, the unit dose is about 60 mg of HC-1119. In yet another embodiment, the dose is about 80 mg of HC-1119. In yet another embodiment, the unit dose is about 100 mg of HC-1119. In yet another embodiment, the unit dose is about 120 mg of HC-1119. In yet another embodiment, the unit dose is about 160 mg of HC-1119. In yet another embodiment, the unit dose is about 200 mg of HC-1119. In some embodiments, the administered unit dosage form contains a crystal form, pharmaceutically acceptable salt, hydrate or solvate or a pharmaceutical composition derived or prepared from the above described amount of the compound of Formula.

In one embodiment, the dosages are administered once a day. In another embodiment, the dosages are administered twice a day. In yet another embodiment, the dosages are administered once every two day. In yet another embodiment, the dosages are administered once every week.

In one embodiment, HC-1119 is administered over a period of at least five (5) weeks. In some embodiments, HC-1119 is administered over a period of at least six (6) weeks or at least eight (8) weeks, or at least ten (10) weeks, or at least twelve (12) weeks.

Another aspect of the invention provides a method of treating an androgen receptor associated disease or condition by administering a subject in need thereof a unit dosage form of the present invention. The disease or condition includes for example prostate cancer, breast cancer, ovarian cancer, acne, pimples, male alopecia, hair loss.

In some embodiments, the unit dosage form contains at least 40 mg, at least 80 mg, at least 100 mg, at least 160 mg or at least 200 mg of the compound of Formula I. In some embodiments, the unit dosage form contains 40 mg, 80 mg, 100 mg, 160 mg or 200 mg of the compound of Formula I. In some embodiments, the administered unit dosage form contains a crystal form, pharmaceutically acceptable salt, hydrate or solvate or a pharmaceutical composition derived or prepared from the above described amount of the compound of Formula I.

In any of the above described methods, the administration schedule may depend on the specific disease or condition. In one embodiment, the unit dosage is administered once a day. In another embodiment, the unit dosage is administered twice a day. In yet another embodiment, the unit dosage is administered once every two day. In yet another embodiment, the unit dosage is administered once every week. In one embodiment, the unit dosage is administered over a period of at least five (5) weeks. In some embodiments, the unit dosage is administered over a period of at least six (6) weeks or at least eight (8) weeks, or at least ten (10) weeks, or at least twelve (12) weeks.

In any of the above described methods, there may include administering, simultaneously with, prior to, or subsequent to the administration of the unit dosage form of the present invention, at least a secondary agent such as PD1 inhibitors, PD-L1 inhibitors, PARP inhibitors, or other AR antagonists. Examples of the PD1 inhibitors and PD-L1 inhibitors include nivolumab (i.e., MDX-1 106, BMS-936558, ONO-4538); CT-011; AMP-224; pembrolizumab; pidilizumab; or MK-3475); BMS936559 (i.e., MDX-1 105); MEDI4736; MSB001071 8C (avelumab); MPDL-3280A); SHR-1210, IBI308, BGB-A317; JS001, GLS-010, GB226, HLX10, AK103, AK104, AK105, AK112, SSI-361, JY034, KN035, SHR1316, TQB2450, KL-A167, CS1001, STI-A1014, JS003, AK106, and HLX-09. Examples of the PARP inhibitors include olaparib, Rucaparib, Niraparib, talazoparib, pamiparip, Zejula, Veliparib, CEP-8983, BGP-15, JPI-289, Fluzoparib, Fuzuopali, BGB-290, ABT-767, MP-124, Simmiparib, Meifupairui, IMP-4297, and WXFL10040340. The dosage and administration schedule of the secondary agent depends on the specific disease or condition and can be determined by one of ordinary skill in the art without undue experiments.

General

As used herein, the term “deuterated” means that hydrogen(s) in a compound or group is substituted by deuterium(s). “Deuterated” can be mono-substituted, bi-substituted, multi-substituted or total-substituted. The terms “one or more deuterium-substituted” and “substituted by deuterium for one or more times” can be used interchangeably.

In one embodiment, the deuterium content in a deuterium-substituted position is greater than the natural abundance of deuterium (0.015%). In some embodiments, the deuterium content in a deuterium-substituted position is greater than 50%, or greater than 75%. In some embodiments, the deuterium content in a deuterium-substituted position is greater than 90%, or greater than 95%, or greater than 97%, or greater than 99%, or greater than 99.5%.

As used herein, the term “compound of the invention” refers to the compound of formula (I). This term also includes various crystal forms, pharmaceutically acceptable salts, hydrates or solvates of the compound of formula (I). As stated above, these terms are used interchangeably herein with “HC-1119” and all refer to 4-{3-[4-cyano-3-(trifluoromethyl)phenyl]-5,5-dimethyl-4-oxo-2-thio-1-imidazolidinyl}-2-fluoro-N-trideuteromethyl benzamide.

As used herein, the term “pharmaceutically acceptable salt” refers to the salts which are suitable for medicine and formed by the compound of the invention with an acid or a base. Pharmaceutically acceptable salts include inorganic salts and organic salts. A preferred salt is formed by the compound of the invention with an acid. The acid suitable for forming salts includes, but not limited to, inorganic acid, such as hydrochloric acid, hydrobromic acid, hydrofluoric acid, sulfuric acid, nitric acid, phosphoric acid; organic acid, such as formic acid, acetic acid, propionic acid, oxalic acid, malonic acid, succinic acid, fumaric acid, maleic acid, lactic acid, malic acid, tartaric acid, citric acid, picric acid, methanesulfonic acid, benzene methanesulfonic acid, benzene sulfonic acid; and acidic amino acid, such as aspartic acid, glutamic acid.

As used herein, the terms “study” and “trial” are used interchangeably to refer to a clinical trial.

As used herein, the terms “Stage” and “Phase” are used interchangeably to refer to the different stages of a clinical trial.

The term “comprising” encompasses “including” as well as “consisting” e.g., a composition “comprising” X may consist exclusively of X or may include something additional e.g., X+Y.

The term “about” in relation to a numerical value x means x±10%.

As used herein, reference to “treatment” of a subject or patient is intended to include prevention, prophylaxis, attenuation, amelioration, alleviation and therapy.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

### EXAMPLES

Examples of embodiments of the present invention are provided in the following examples. The following examples are presented only by way of illustration and to assist one of ordinary skill in using the invention. The examples are not intended in any way to otherwise limit the scope of the invention. Further, it should be understood that in the present invention, the technical features specifically described above and below can be combined with each other, thereby constituting a new or preferred technical solution, which needs not be specified.

### Example 1. Preparation of the Compound of the Invention

The preparation of HC-1119 of the present invention is described in detail below. However, these specific methods are not provided for the limitation of the invention. The compound of the invention can be readily prepared by optionally combining any of the various methods described in the specification or various methods known in the art, and such combination can readily be carried out by one skilled in the art.

In general, during the preparation, each reaction is conducted in solvent, at a temperature between room temperature to reflux temperature (such as 0-120° C., preferably 0-80° C.). Generally, the reaction time is 0.1-60 hours, preferably, 0.5-48 hours.

In one embodiment, the preparation method for HC-1119, 4-{3-[4-cyano-3-(trifluoromethyl)phenyl]-5,5-dimethyl-4-oxo-2-thio-1-imidazolidinyl}-2-fluoro-N-trideuteromethylbenzamide is as follows:

### Synthesis of 2-fluoro-N-trideuteromethyl-4-nitro-benzamide (compound B)

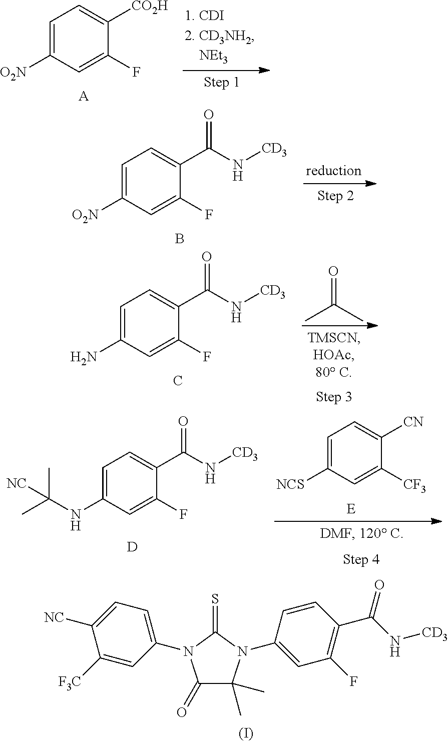

Into a solution of compound A (5.25 g, 28.37 mol) in dichloromethane (20 mL) CDI (4.62 g, 28.37 mmol) was added. The reaction mixture was stirred at room temperature for one hour. Into a solution of trideuterated methylamine hydrochloride (2 g, 28.76 mmol) in methylene chloride (20 mL) triethylamine (3.27 g, 32.36 mmol) was added to give a white suspension. After stirred at room temperature for half an hour, the suspension was added to the reaction mixture slowly. The resulting mixture was stirred for another hour, the reaction was quenched by adding water (10 mL). The organic phase was separated and the aqueous phase was extracted with dichloromethane (2×20 mL) twice. The organic phases were combined, washed with 1 M hydrochloric acid (2×10 mL) twice, 1 M aqueous sodium hydroxide solution (2×10 mL) twice and saturated brine (10 mL) once, dried (Na₂SO₄), filtered and concentrated under reduced pressure to give a white solid compound B (compound B, 5.1 g, 88.2% yield); MS: 202 (M+H⁺).

### Synthesis of 4-amino-2-fluoro-N-trideuteromethyl benzamide (Compound C)

Compound B (5.1 g, 25.37 mmol) was dissolved in EtOAc and acetic acid (15 mL+5 mL) to form a solution. Then to this solution was added iron powder (15 g) and the reaction refluxed overnight for 16 h. The reaction was cooled to room temperature and precipitate was filtered and washed with EtOAc 3 times (3×20 mL). The organic solutions were combined, washed with brine, dried over sodium sulfate, concentrated to give yellow solid. It was further purified by flash chromatography (DCM:MeOH=50:1) to give compound C as a pale yellow solid (2.22 g, 51.2% yield). ¹H NMR (CDCl₃, 400 MHz): δ (ppm) 7.92 (1H, t, J=8.8 Hz), 6.59 (1H, s), 6.49 (1H, d, J=8.4 Hz), 6.32 (1H, d, J=14.4 Hz), 4.10 (2H, s).

### Synthesis of 4-(2-cyano-2-propylamino)-2-fluoro-N-trideuteromethyl-benzamide (Compound D)

TMSCN (4 g, 40.3 mmol) and compound C (1.5 g, 8.76 mmol) were dissolved in a mixed solution of acetic acid (10 mL) and acetone (10 mL). The resulting mixture was maintained in a sealed tube at 80° C. overnight (16 h), and then cooled to room temperature. Acetone was removed under reduced pressure, water was added (20 mL), and the resulting mixture was extracted with ethyl acetate, washed with brine, dried over sodium sulfate, and concentrated. The resulting solid was washed with petroleum ether (10 mL) and dried by suction to give compound D as a white solid (1.95 g, 93.4% yield).

### Synthesis of 4-{3-[4-cyano-3-(trifluoromethyl)phenyl]-5,5-dimethyl-4-oxo-2-thio-1-imidazolidinyl}-2-fluoro-N-trideuteromethyl benzamide (HC-1119)

Compound D (0.5 g, 2.1 mmol) and compound E (0.5 g, 2.19 mmol) were dissolved in DMF (10 mL). The resulting mixture was heated to 120° C. overnight (16 h). Ethanol (5 mL), water (5 mL) and concentrated hydrochloric acid (1 mL) were added, and the resulting mixture was heated at reflux for 1 h. The mixture was extracted with ethyl acetate, washed with brine, dried over sodium sulfate, and concentrated. The residue was purified by column chromatography (PE:EA/1:1) to give a brown solid which was further purified by preparative chromatography to give compound (I), HC-1119 as a brown solid (132.7 mg, 11% yield). ¹H NMR (DMSO-d₆, 400 MHz): δ (ppm) 8.44 (1H, s), 8.41 (1H, d, J=8.4 Hz), 8.30 (1H, s), 8.09 (1H, d, J=7.6 Hz), 7.79 (1H, t, J=8 Hz), 7.44 (1H, d, J=11.2 Hz), 7.34 (1H, d, J=8.8 Hz), 1.54 (6H, s). MS: 477.2 (M+H⁺).

### Example 2: Description of Open-Label Clinical Trial to Evaluate Tolerability, Pharmacokinetics and Efficacy in Patients with Metastatic Castration-Resistant Prostate Cancer

An open-label study of soft gel capsules of HC-1119 was conducted on patients with metastatic castration-resistant prostate cancer (mCRPC). There were two (2) stages or arms to the study, Stage A and Stage B.

Stage A had four (4) dose-escalation cohorts of 40 mg, 80 mg, 160 mg, and 200 mg of HC-1119. During Stage A, the dose escalation tolerance (or tolerability) study was conducted using the traditional rule-based 3+3 design. All subjects in Stage A were involved in pharmacokinetic studies. Cohorts 80 mg and 160 mg were each increased to 8 subjects to participate in the PK study. Table 1 provides a summary of Stage A.

TABLE 1

| | | | Dose | Increased cases | |
|---|---|---|---|---|---|
| Cohort | Drug | Dose | Escalation | for PK study | Total |
| 1 | HC-1119 | 40 mg | 3 | 0 | 3 |
| 2 | HC-1119 | 80 mg | 3 | 5 | 8 |
| 3 | HC-1119 | 160 mg | 3 | 5 | 8 |
| 4 | HC-1119 | 200 mg | 3 | 0 | 3 |
| 5* | HC-1119 | 120 mg | 3 | 5 | 8 |

*If Dose Limiting Toxicity (DLT) occurred in ≥2/6 subjects in the 160 mg dose
group, the dose will be reduced to 120 mg for tolerance and pharmacokinetic studies.

### Example 3. Study Objectives

The primary outcome measures were to evaluate the tolerability and safety of HC-1119 in patients with mCRPC and to explore the dose-limiting toxicity (DLT).

The secondary outcome measures were to assess the pharmacokinetic (PK) parameters of HC-1119 and to assess the pharmacodynamics characteristics of HC-1119 on serum PSA.

### Example 4. Dose-Escalation Regimen

Starting Dose Estimation:

The similar drug enzalutamide had good safety results in the dose range of 30 mg-240 mg, with the maximum human exposure being 600 mg and DLT occurring at the dose of 360 mg. With enzalutamide, the initial dose in the human trials was 30 mg of enzalutamide.

Preclinical studies have shown that, under similar exposures, the toxicological characteristics and the target organ of HC-1119 are similar to those of enzalutamide.

The highest non-severely toxic dose (HNSTD) for beagle dogs in repeated dose toxicity tests (28 days) was 90 mg/kg. In this conversion, adult equivalent dose (HED) was calculated to be: 0.54*90 mg/kg*60 kg=2916 mg/d. General antineoplastic drugs in the human body are safe at an initial dose of ⅙ HNSTD. This set the initiator at 486 mg/day. This was then divided by an additional safety factor of 12, setting the starting dose at set at 40 mg/day of HC-1119.

The maximum tolerable dosage (MTD) in the Phase I dose escalation study of the similar drug, enzalutamide, was 240 mg/d. A fifth (⅕) of that provides a dose of 48 mg/d.

The clinical treatment dose during the study with the similar drug, enzalutamide, was 160 mg/d. Pre-clinical pharmacodynamics studies showed that the activity of HC-1119 was better than that of enzalutamide. Choosing the initial dose of 40 mg/d was safe and reduced the risk of exposure to ineffective doses. After comprehensive safety and ethical considerations, 40 mg/d was chosen to be the initial dose of the dose-escalation test.

The Maximum Dose:

Preclinical efficacy studies showed that HC-1119 activity was superior to the similar drug, enzalutamide. The maximum tolerated dose of enzalutamide was 240 mg/d and its clinical dosage was 160 mg/d. Therefore, the maximum dose of this study was designed to 200 mg/d.

Based on the test results, if more than 200 mg needed to be further explored, the researchers and the sponsor were to discuss whether to continue the dose escalation tolerance study in the higher dose group.

Dose-Escalation Rules:

The following were the dose-escalation rules that were followed during this study/trial.
(1) After the initial dose, according to the modified Fibonacci method, the dose was carried out from low to high, which were respectively 80 mg, 160 mg and 200 mg dose groups, and the subjects were enrolled in the traditional 3+3 mode.
(2) Each dose group first enroll three subjects. Every subject was observed after a single administration of HC-1119 for 24 hours. If no DLT appeared, then the subject entered the continuous administration period. If no DLT occurred in the 3 enrolled subjects, additional subjects were enrolled to a higher dose group. If DLT was present in 1 of the 3 enrolled subjects, then 3 subjects were re-enrolled in this dose group, and if no DLT was seen in the 3 re-enrolled subjects, then subjects were enrolled in the next higher dose group.
(3) If there were greater than 2/6 subjects with DLT in the 160 mg dose group, the dose was reduced to 120 mg for the tolerability study.

### Example 5. Dose-Limiting Toxicity (DLT)

### Definition

Adverse events are those that occur during the dosing period (within 42 days) that may be associated with HC-1119 and meet any of the following criteria according to the NCI-CTC AE 4.03 grading criteria, will be considered DLT:

(1) Grade III/IV epilepsy, spasticity, or Grade II epilepsy (brief generalized seizures) in which case administration of HC-1119 was stopped, based on the judgment of the investigators;

(2) Hematological toxicity: Degree IV drug-related hematology AEs; Degree III neutropenia accompanied by significant fever (higher than 38.5° C.);

(3) Non-hematological toxicity: Degree III/IV drug-related AEs;

(4) Hair loss, Degree III/IV fever, nausea, vomiting, fatigue that were not accompanied with neutropenia were not considered as DLT; and

(5) Any level of toxicity that required termination of treatment, based on the judgment of the investigator and the sponsor.

### Example 6. Preliminary Efficacy Evaluation

Stage A: PSA was dynamically observed to assess the initial dose-effectiveness based on data from subjects in each dose group until the end of the 12-week trial period. Serum PSA test, safety evaluation (including blood, liver and kidney function, electrocardiogram, etc.) were performed at the 6th, 8th, 10th and 12th weeks of visit, and the trial ended at the 12th week of visit.

Stage B: A dynamic PSA study was conducted to assess the initial dose-effectiveness of each dose group based on data from the subject's use of the test drug until the 12-week ‘weekend. Serum PSA testing was performed at Weeks 3, 6, and 12 and ended at Week 12 of visit.

### Example 7. Pharmacokinetic Studies

All subjects in Stage A were involved in pharmacokinetic studies, and for the Dose Group of 80 mg and 160 mg each group was increased to 8 subjects to involve in the PK study. If there were more than 2/6 subjects with DLT in the 160 mg dose group, the dose was reduced to 120 mg for tolerability studies. If the 120 mg dose was tolerable, then the number of subjects was increased to 8 cases for PK study.

Single Dose Biological Sample Collection:

Subjects were given a single fasting dose on the first day of the study. Blood samples were collected before (−0.5˜0 h) and after administration 0.5, 0.75, 1, 2 h (±5 min), 4, 8, 12 h (±15 min), 24 h (±30 min, Day 2 before administration). Venous blood (3 mL) was collected, set in an anticoagulant tube (type of anticoagulant was based on established analytical test methods).

Continuous Administration of Biological Samples Collected:

Blood sample collected on continuous administration Day 7, 21, 35, 42, 56, 70, 84 before administration, on Day 84 after administration 0.5, 0.75, 1, 2 h (±5 min), 4, 8, 12 h (±15 min), 24 h (±30 min, Day 85 before administration). Venous blood (3 mL) was collected, set in an anticoagulant tube (type of anticoagulant was based on established analytical test methods).

According to the observed PK data, determined by the researcher and the sponsor, the specific time, frequency and quantity of PK sample collection was adjusted.

Sample Handling:

Samples were centrifuged (about 3000 rpm for 10 minutes) to separate the plasma as soon as possible. Isolated plasma samples were stored in duplicate in plasma cryostat, with the tagged population's enrollment number, subject's initials, date and time of collection. Plasma samples were stored in −60° C.˜−80° C. freezer as soon as possible. One blood sample was retained at the research center and one blood sample was shipped to a specialist testing company for plasma drug concentration determination. Compounds tested included HC-1119 prototype (M0) and metabolites M1, M2.

### Example 8. Eligibility Criteria

Eligible subjects needed to have met/provided all of the following criteria:

(1) Willing and able to provide informed consent;
(2) Men, 18 years of age or older;
(3) Histologically or cytologically confirmed adenocarcinoma of the prostate, without neuroendocrine carcinoma or ductal adenocarcinoma;
(4) Evidence of distant metastatic disease (such as bone scans and CT/MRI findings);
(5) Castration (surgery or drugs), or combination with androgen deprivation therapy were relapsed, invalid, or advanced (disease progression was defined as one or more of the following three episodes occurred in the subject: (a) PSA progression, defined as a continuous increase in PSA levels of at least 3 measurements (≥1 week interval), with a>50% increase from the lowest and PSA levels>2 ng/mL at enrollment; (b) Progression of soft tissue disease as defined by RECIST 1.1; (c) Bone disease progression as defined by PCWG2, two or more new lesions found on bone scans);
(6) Total testosterone <50 ng/dl;
(7) Subjects who had not undergone bilateral orchidectomy must maintain effective GnRH analogue treatment throughout the study;
(8) Estimated survival time >6 months;
(9) ECOG performance status ≤1;
(10) Laboratory tests meet the following criteria: (a) Blood tests: hemoglobin (Hb) ≥90 g/L (no blood transfusion within 14 days); Absolute Neutrophil Count (ANC) ≥1.5×10⁹/L; PLT ≥80×10⁹/L; (b) Blood biochemical tests: Serum creatinine (Cr) ≤2×upper limit of normal (ULN), or creatinine clearance (CrCl) calculated ≥60 mL/min when serum creatinine>2×upper limit of normal (ULN) in subjects. Bilirubin BIL≤2×ULN. Alanine aminotransferase (ALT), aspartate aminotransferase (AST)≤2.5×ULN (subjects with liver metastases ≤5×ULN); and (c) Coagulation function: International normalized ratio (INR)<1.5.

### Example 9. Subject Exclusion Criteria

Subjects were ineligible for inclusion, or were excluded from the study if they met any one of the following criteria:

1) Did not recover after a toxicity of Grade 2 or above after original treatment;
2) Clinically significant gastrointestinal abnormalities that could affect drug intake, transport or absorption (e.g., inability to swallow, chronic diarrhea, intestinal blockage, etc.) or total gastrectomy;
3) Allergies, or known to have a history of allergy to the drug components;
4) Metastases in the brain;
5) History of another malignancy within the previous 5 years (except for non-melanoma skin cancers that had been cured);
6) History of organ transplant;
7) HIV antibody positive;
8) Previous history of epilepsy or severe central nervous system disease history,
9) Unexplained coma history;
10) Family history of epilepsy;
11) History of traumatic brain injury,
12) History of drug abuse;
13) Serious cardiovascular disease, myocardial infarction, or arterial thrombosis, or unstable angina in the past 6 months, or in patients with clinical symptoms of heart failure;
14) Poorly controlled hypertension (systolic blood pressure ≥160 mmHg or diastolic blood pressure ≥100 mmHg), patients with a history of hypertension were allowed to participate in the study if their blood pressure was controlled by antihypertensive therapy;
15) Must combine with the use of drugs known to reduce the threshold of seizures during the trial;
16) Received 5α-reductase inhibitors (Finasteride, Dutasteride), Estrogen or Cyproterone treatment within 4 weeks;
17) Received ketoconazole treatment within 4 weeks;
18) Previously used investigational drugs or listed drugs that blocked androgen synthesis (such as Abiraterone Acetate, TAK-683, TAK-448) or against androgen receptors (such as enzalutamide, SHR3680, GT0918 (Proxalutamide), ARN509);
19) Participated in other drug clinical trials within 1 month prior to enrollment; or
20) Unsuitable to participate in this study as determined by the researchers.

### Example 10. Results of HC-1119 Phase 1a Studies

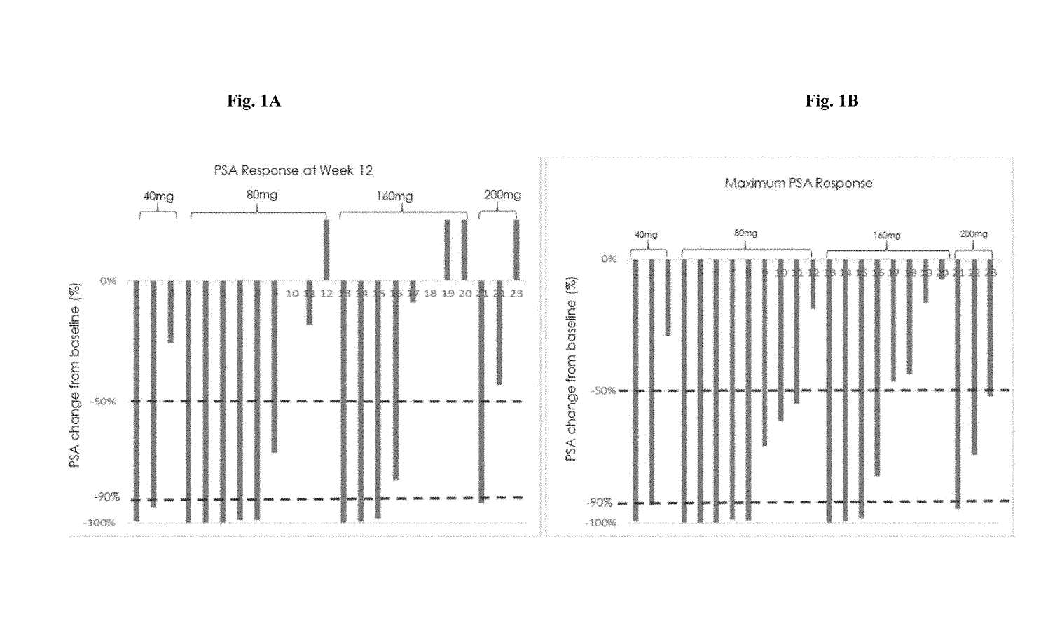

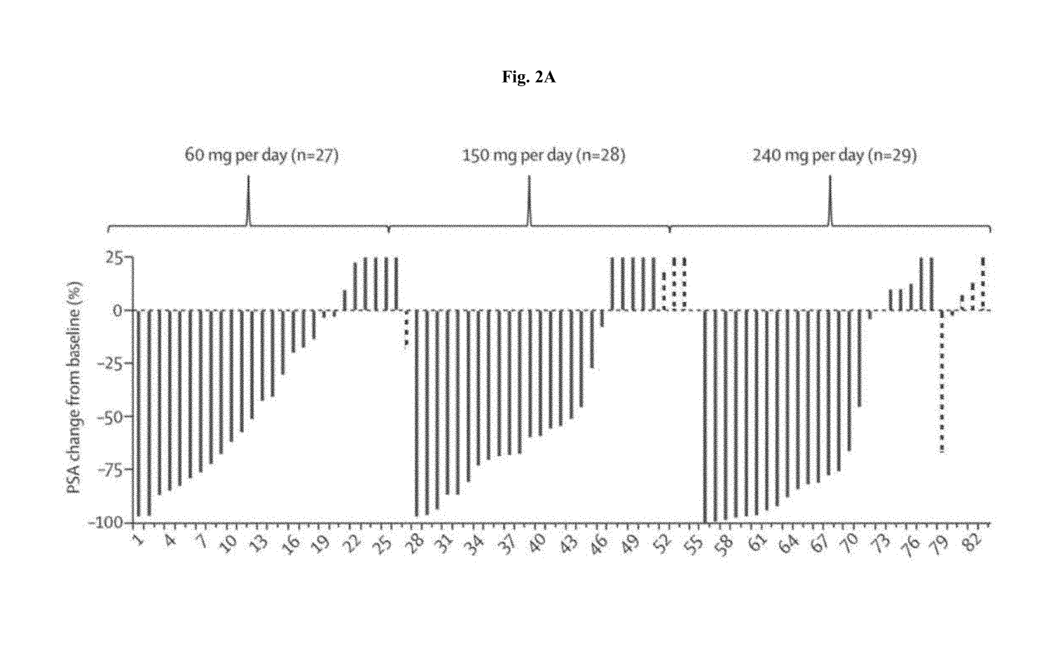

FIGS. 1A and 1B of FIG. 1 show the results of the HC-1119 Phase 1a clinical studies. They show the percentage change in PSA levels for subjects in the study. In both figures, the vertical axis represents the percent change in PSA levels from the baseline and the horizontal axis (numbers that are in line with the “0%”) identifies individual patient or subject numbers.

FIG. 1A shows the response rate at week 12 of the study. The rates of descent or decrease in PSA levels in the dose groups 40 mg, 80 mg, 160 mg and 200 mg by more than 50% from the baseline were ⅔ (66.7%), 6/8 (75%), 4/7 (57.1%), and ⅓ (33.3%), respectively. Two (2) patients, one in the dose group 80 mg and another in the dose group 160 mg, did not complete the trial.

FIG. 1B shows the maximum response rate for subjects in the study. The maximum descent or decrease in PSA levels in the dose groups 40 mg, 80 mg, 160 mg and 200 mg by more than 50% from the baseline were ⅔(66.7%), 8/9 (88.8%), 4/8 (50%) and 3/3 (100%), respectively.

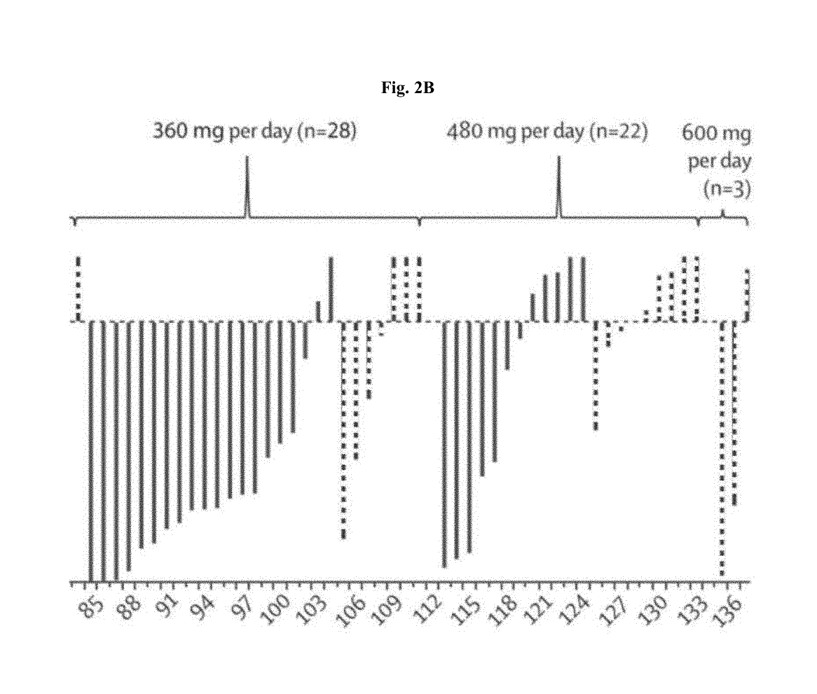

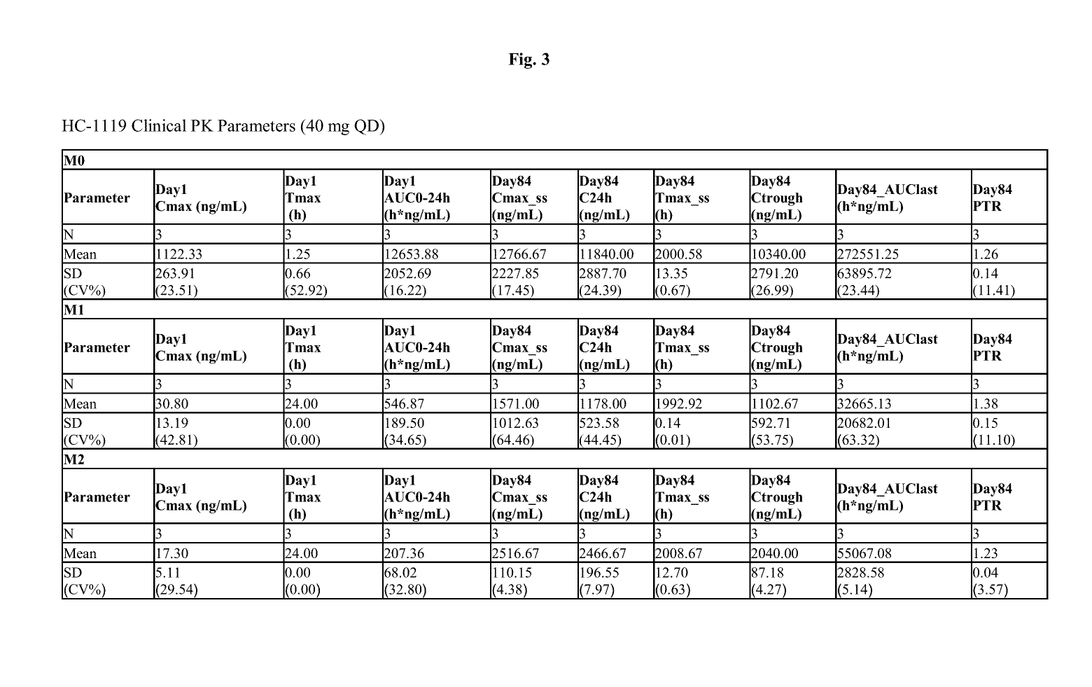

FIGS. 2A and 2B show the results of the Phase I/II studies with enzalutamide. FIGS. 2A and 2B show the percentage change in PSA levels for subjects in the study. In both figures, the vertical axis represents the percent change in PSA levels from the baseline and the horizontal axis identifies individual patient numbers. The solid lines show the decrease in PSA levels at 12 weeks for patients who stayed on treatment for at least 12 weeks; the dashed lines show decrease at less than 12 weeks for patients who did not stay on treatment for at least 12 weeks.

FIG. 2A shows the results for subjects dosed with 60 mg/day (27 subjects), 150 mg/day (28 subjects), and 240 mg/day (29 subjects). FIG. 2B shows the results for subjects dosed with 360 mg/day (28 subjects), 480 mg/day (22 subjects), and 600 mg/day (3 subjects).

### Example 11. Inter-Patient Variability is Reduced with HC-1119

As provided above, inter-patient variability in the plasma concentrations and/or AUC of subjects administered with HC-1119 is reduced in comparison with those that receive the non-deuterated drug, enzalutamide.

FIG. 3 provides the clinical PK parameters (40 mg QD) from the HC-1119 trial.

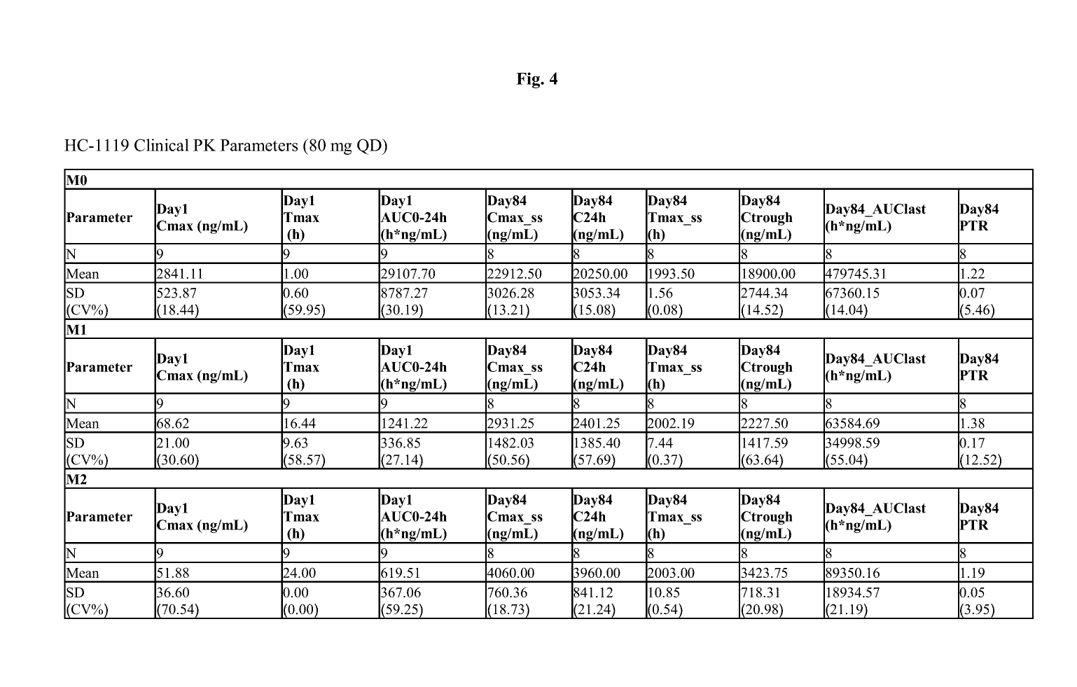

FIG. 4 provides the clinical PK parameters (80 mg QD) from the HC-1119 trial.

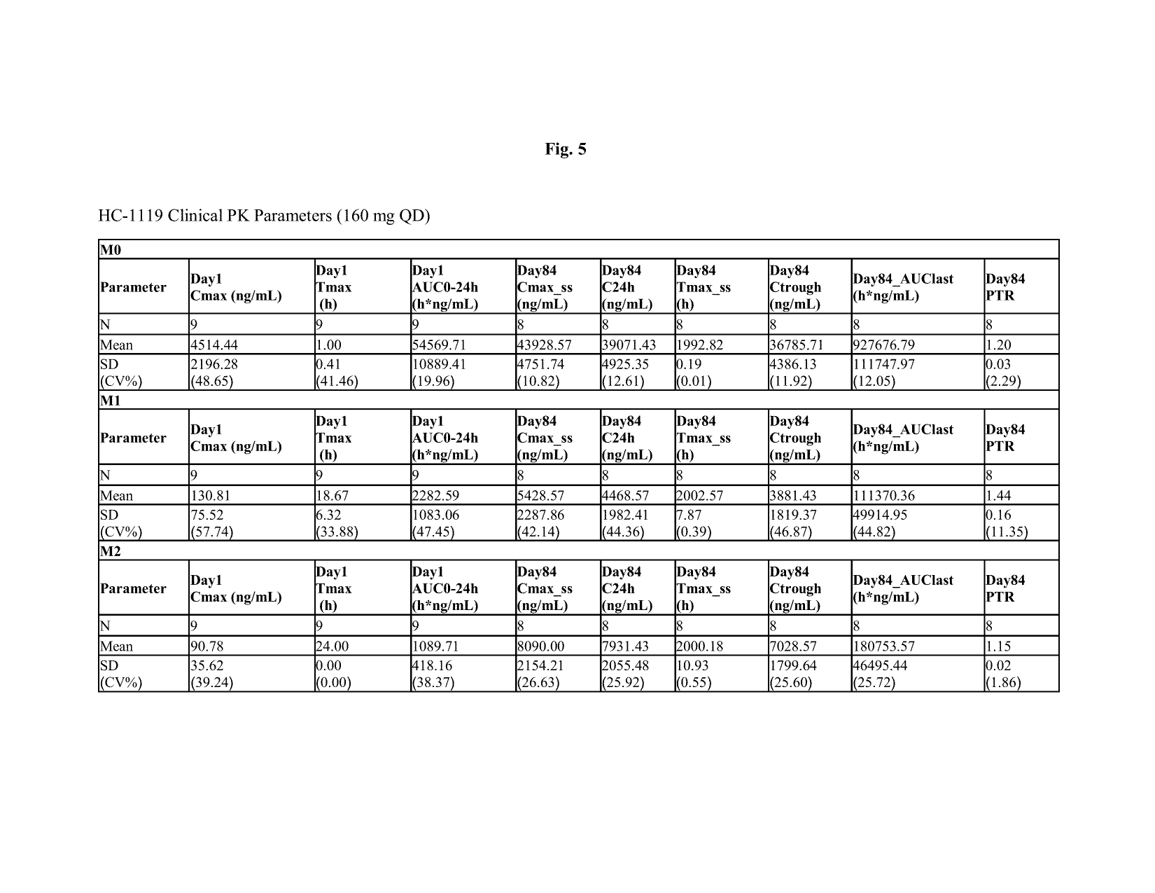

FIG. 5 provides the clinical PK parameters (160 mg QD) from the HC-1119 trial.

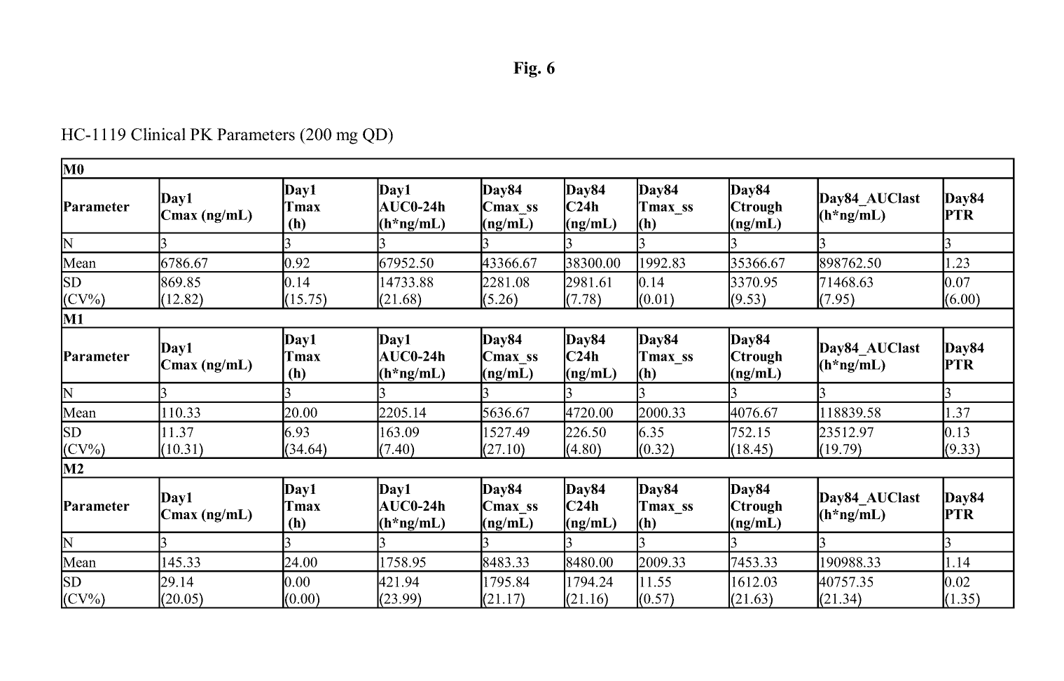

FIG. 6 provides the clinical PK parameters (200 mg QD) from the HC-1119 trial.

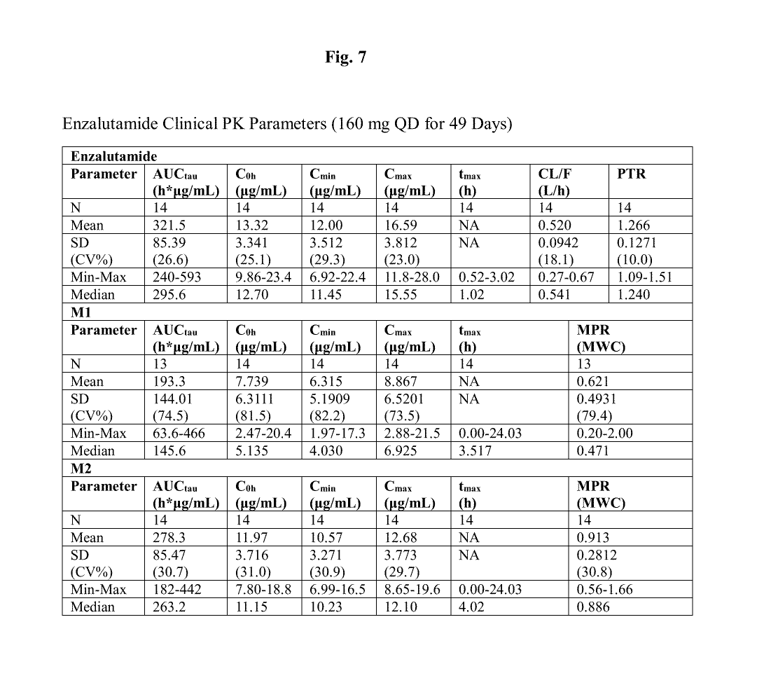

FIG. 7 provides the clinical PK parameters from an enzalutamide study. The data was obtained from FDA NDA documents and study number 9785-CL-0007.

The extent of inter-patient variation or variability was measured by the coefficient of variation (CV %). It can be seen from the tables provided in FIGS. 3-7 that HC-1119 demonstrated reduced inter-patient variation compared to enzalutamide after reaching steady state. FIG. 7 shows steady state clinical PK parameters of enzalutamide (160 mg QD for 49 days). For M0, the CV for Cₘₐₓ, Cₘᵢₙ, and AUCₜₐᵤ are 23.0%, 29.3% and 26.6%, respectively. At similar exposure, they are best compared with HC-1119 at 80 mg group. As shown in FIG. 4 (HC-1119, 80 mg group clinical PK), for HC-1119-M0, the CV for Day 84 Cₘₐₓ, C_{trough}, and AUCₗₐₛₜ are 13.21%, 14.52% and 14.04%, respectively. On all three measurements, HC-1119-M0 demonstrated lower inter-patient variation. This is also the case for HC-1119-M1 and HC-1119-M2. For enzalutamide M1, the CV for steady state Cₘₐₓ, Cₘᵢₙ, and AUCₜₐᵤ are 73.5%, 82.2% and 74.5%, respectively, while for HC-1119-M1, the CV for steady state Cₘₐₓ, C_{trough}, and AUCₗₐₛₜ are 50.56%, 63.64% and 55.04%, respectively. For enzalutamide M2, the CV for steady state Cₘₐₓ, Cₘᵢₙ, and AUCₜₐᵤ are 29.7%, 30.9% and 30.7%, respectively, while for HC-1119-M2, the CV for steady state Cₘₐₓ, C_{trough}, and AUCₗₐₛₜ are 18.73%, 20.98% and 21.19%, respectively. The same conclusion can also be made for other HC-1119 dose groups by comparing the CV in FIG. 3 (HC-1119 40 mg), FIG. 5 (HC-1119 160 mg) and FIG. 6 (HC-1119 200 mg) with those in FIG. 7 (enzalutamide 160 mg). HC-1119 demonstrated reduced inter-patient variation or variability, and thus has better safety accordingly.

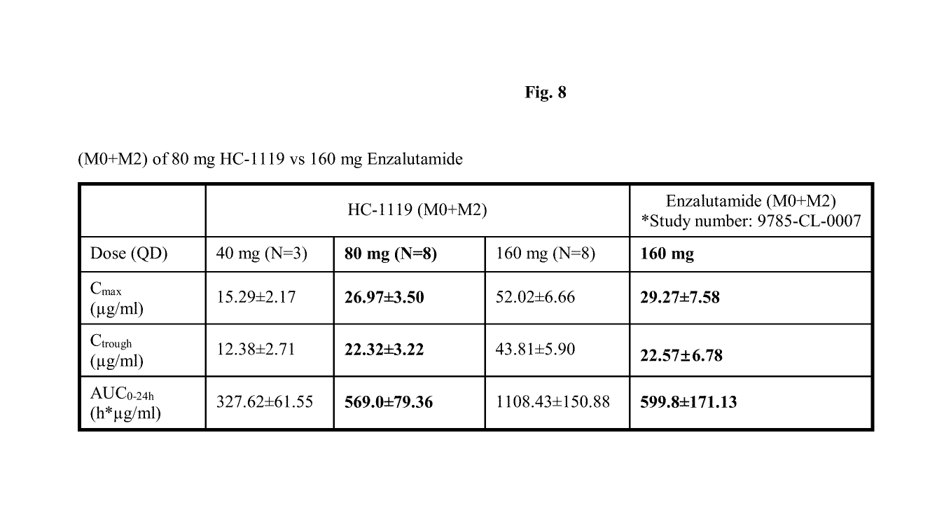

FIG. 8 provides some PK parameters for 80 mg HC-1119 in comparison with those of 160 mg. of the non-deuterated drug, enzalutamide. The tables show that at steady state, the parameters for active plasma drug compounds (M0+M2) of 80 mg HC-1119 is close to those obtained at double the dosage, 160 mg, of enzalutamide in terms of Cₘₐₓ, C_{trough}, and AUC₀₋₂₄ₕ.

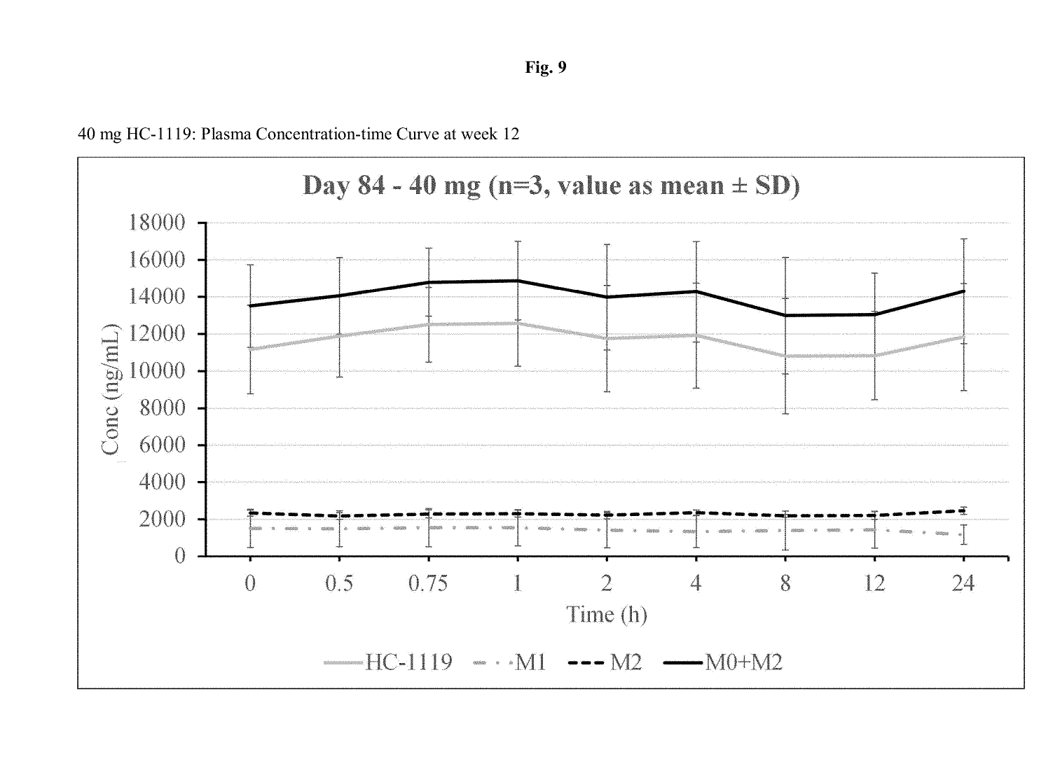

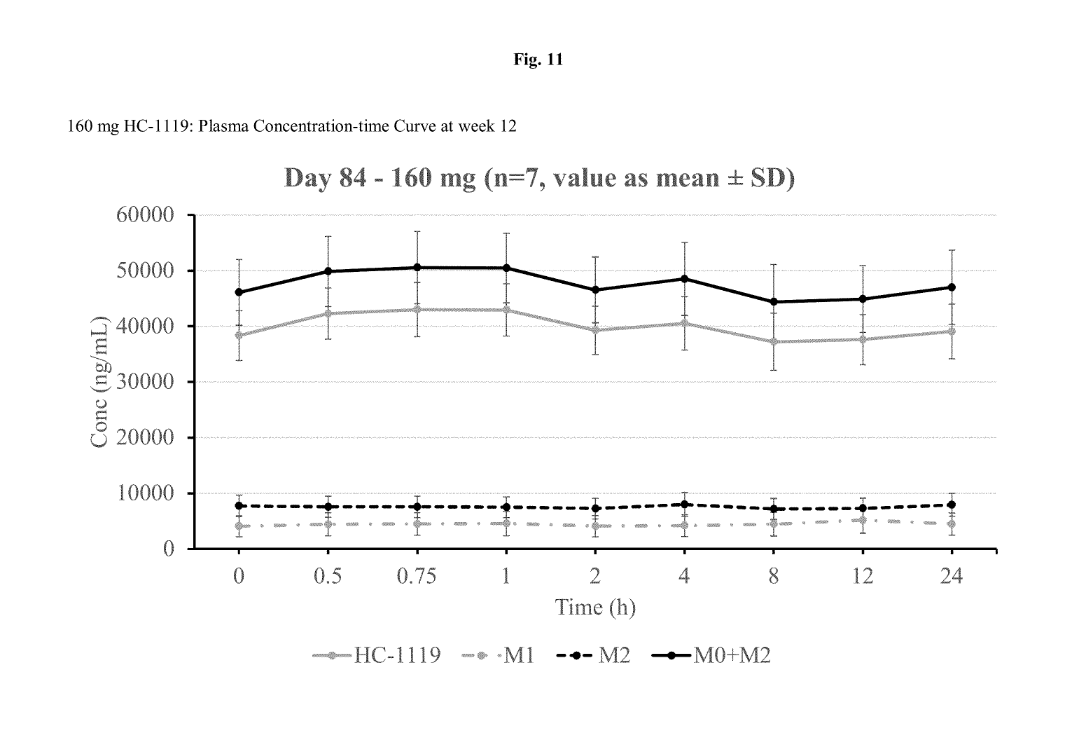

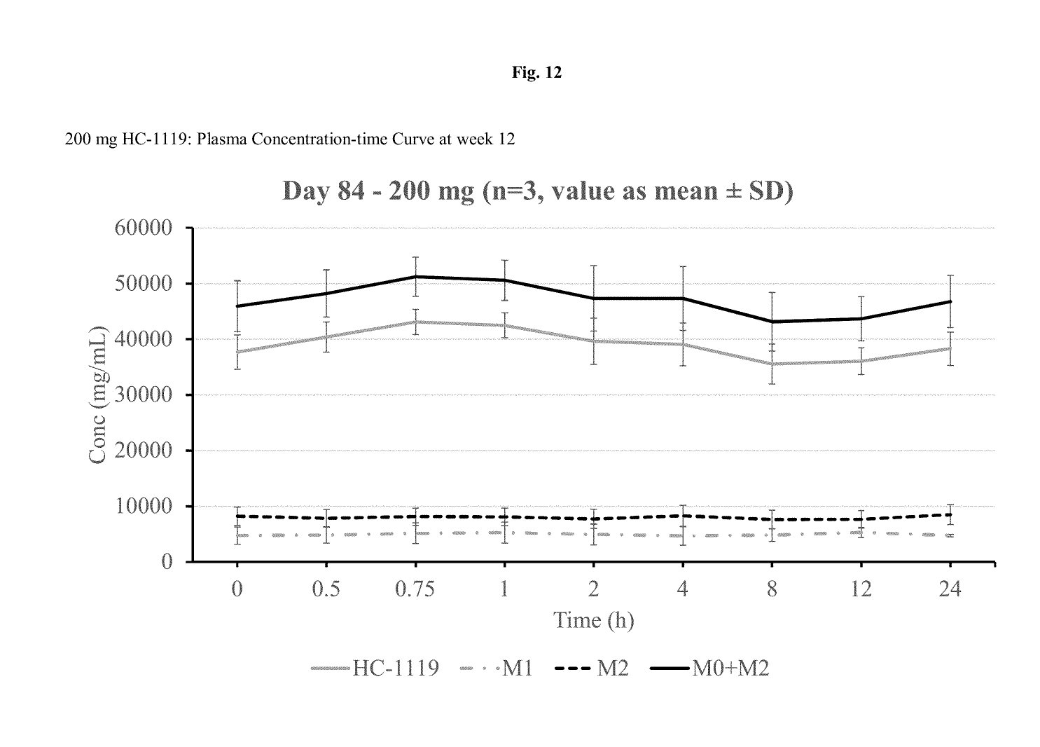

FIGS. 9-12 show the plasma concentration versus time Curve at week 12 for patients dosed with 40 mg, 80 mg, 160 mg and 200 mg, respectively, of HC-1119.

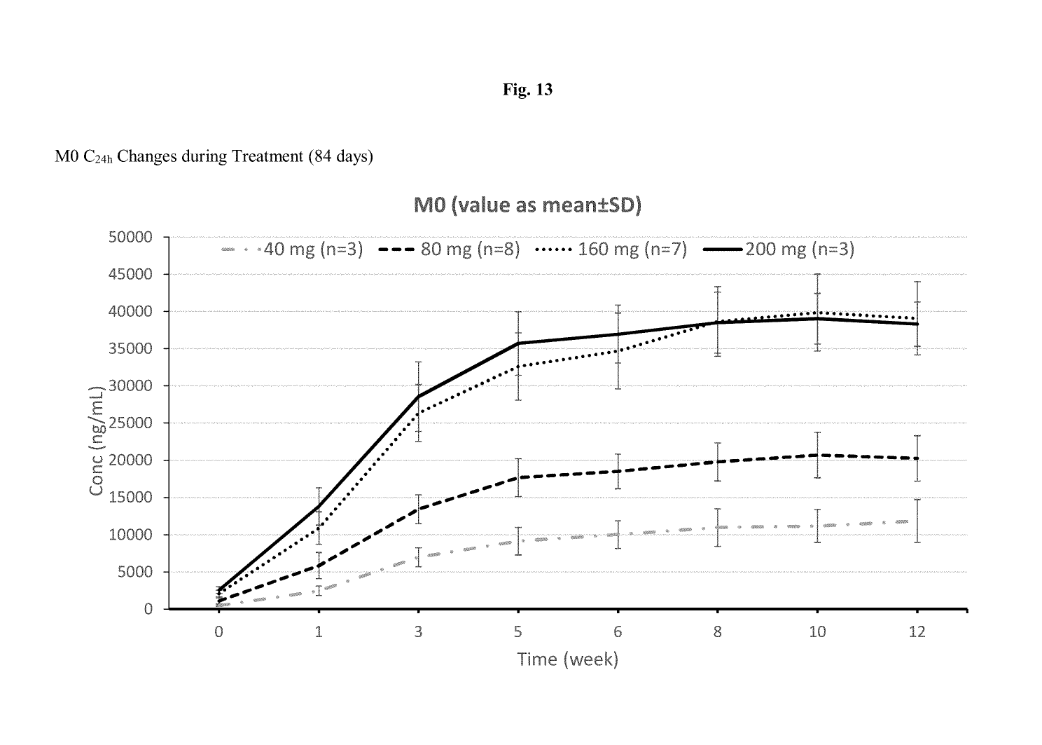

FIG. 13 shows the changes in C₂₄ₕ versus time for the parent drug (HC-1119-M0) during the treatment period of the trial (84 days) for patients dosed with 40 mg, 80 mg, 160 mg and 200 mg of HC-1119. It depicts the M0 concentration values as the mean±SD.

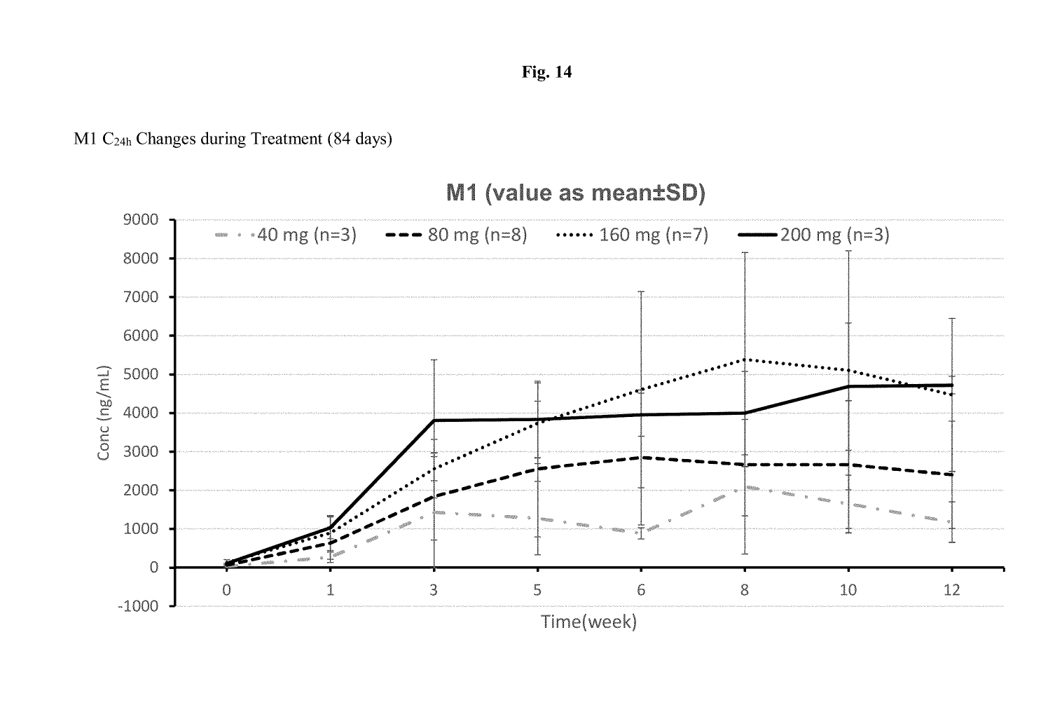

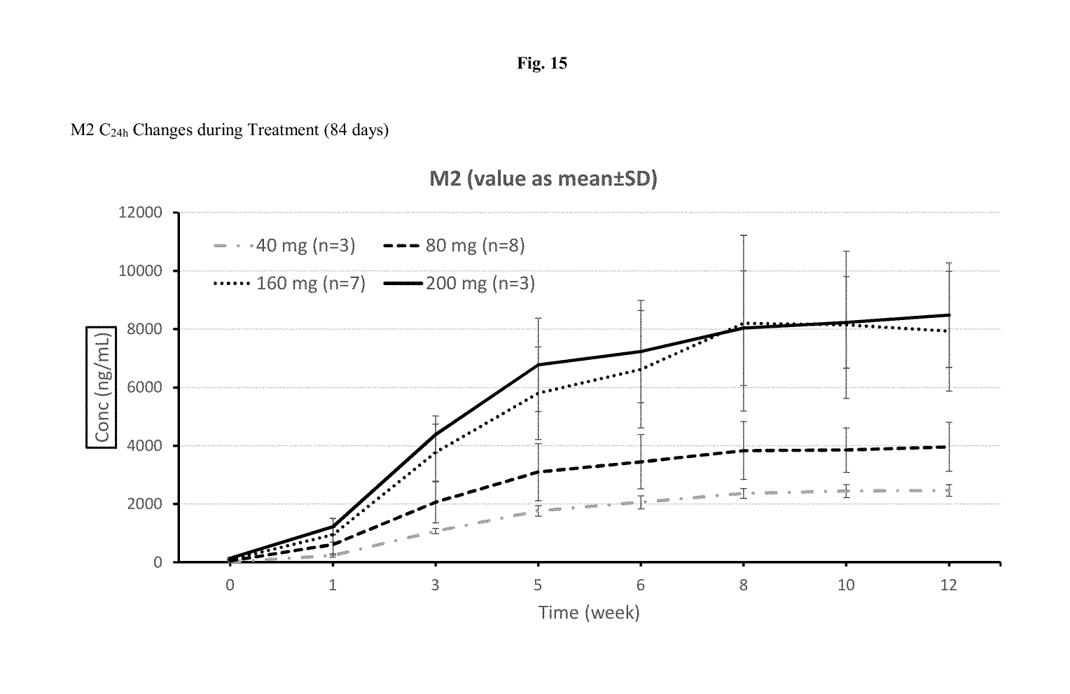

FIGS. 14 and 15 show the changes in C₂₄ₕ versus time for the first and second metabolites, M1 and M2, respectively, during the treatment period of the trial (84 days) for patients dosed with 40 mg, 80 mg, 160 mg and 200 mg of HC-1119. They depicts the M0 concentration values as the mean±SD.

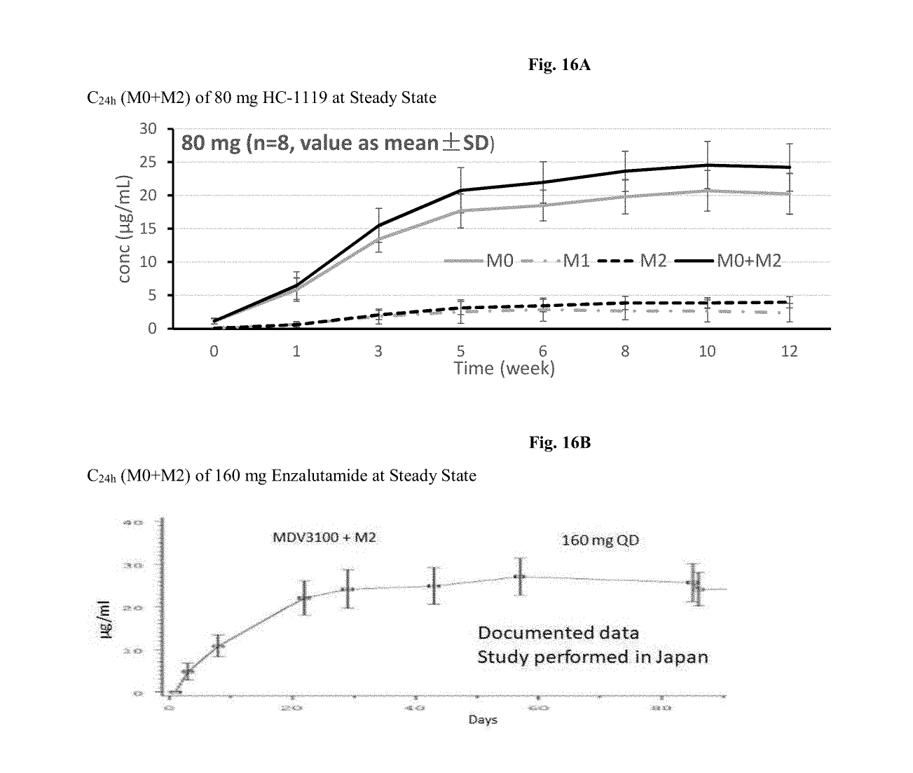

FIGS. 16A and 16B show the changes in C₂₄ₕ versus time for the combination of the parent drug and its active, second metabolite (HC-1119 M0+M2) at steady state during treatment for patients dosed with either 80 mg HC-1119 (FIG. 16A) or 160 mg enzalutamide (FIG. 16B). As can be observed, the C₂₄ₕ for the combination of the active drugs (HC-1119 M0+M2) of 80 mg HC-1119 is close to that of 160 mg enzalutamide at steady state.

### Example 12. HC-1119 has Higher Safety Margin

As discussed above, enzalutamide (M0) has two major metabolites, inactive M1 and active M2. Deuteration stabilizes M0 by blocking the generation of M1 and M2. Also, enzalutamide has the risk of inducing epilepsy due to the brain penetration of M0 and M2.

Shown below is the pathway by which M1 and M2 are generated for HC-1119 and enzalutamide, respectively.

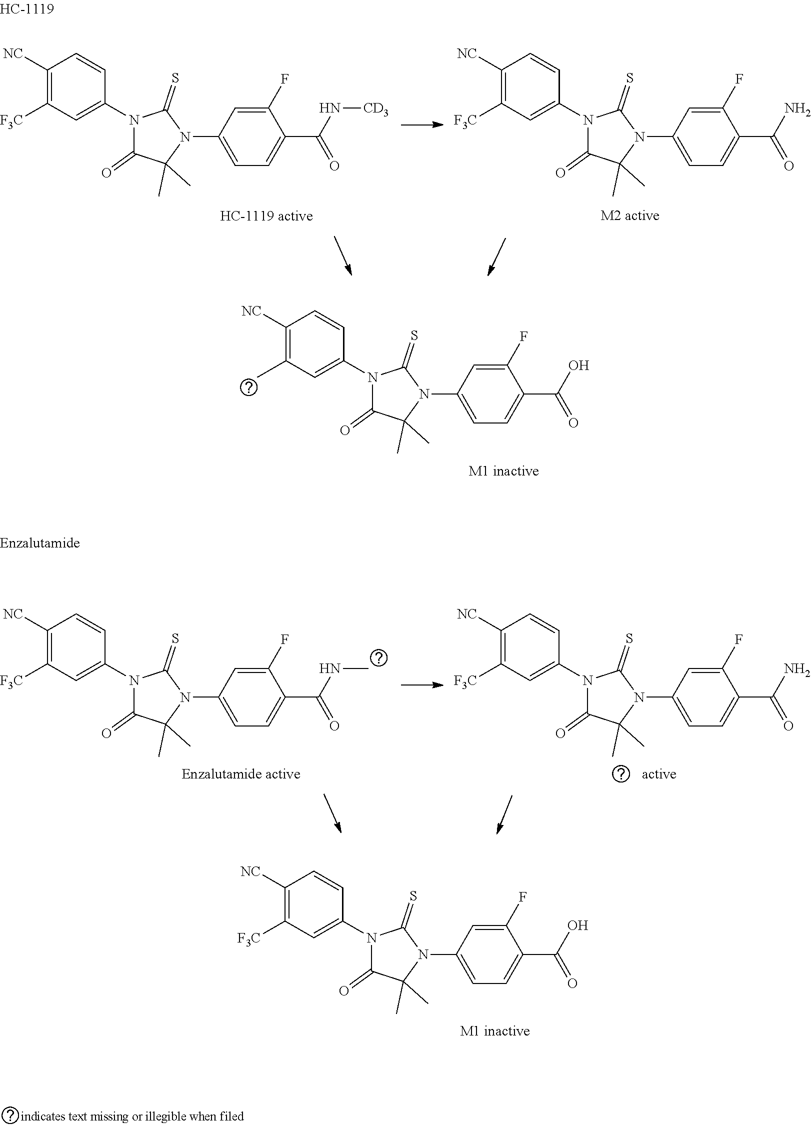 [SMILES: [2H]C([2H])([2H])NC(=O)c1ccc(N2C(=S)N(c3ccc(C#N)c(C(F)(F)F)c3)C(=O)C2(C)C)cc1F ; CC1(C)C(=O)N(c2ccc(C#N)c(C(F)(F)F)c2)C(=S)N1c1ccc(C(N)=O)c(F)c1 ; Cc1cc(N2C(=O)C(C)(C)N(c3ccc(C(=O)O)c(F)c3)C2=S)ccc1C#N ; CNC(=O)c1ccc(N2C(=S)N(c3ccc(C#N)c(C(F)(F)F)c3)C(=O)C2(C)C)cc1F ; CC1(C)C(=O)N(c2ccc(C#N)c(C(F)(F)F)c2)C(=S)N1c1ccc(C(N)=O)c(F)c1 ; CC1(C)C(=O)N(c2ccc(C#N)c(C(F)(F)F)c2)C(=S)N1c1ccc(C(=O)O)c(F)c1]

M1 is inactive; M0 and M2 are both active with similar activities (for AR binding, M0 Ki=51 nM, M2 Ki=57 nM; for LNCaP growth inhibition, M0 IC50=0.12 nM, M2 IC50=0.13 nM). In patients, at steady state of 160 mg/day of enzalutamide, the C_{trough} ratio of M0:M2:M1 is 1:1:0.5 (obtained from FDA documents).

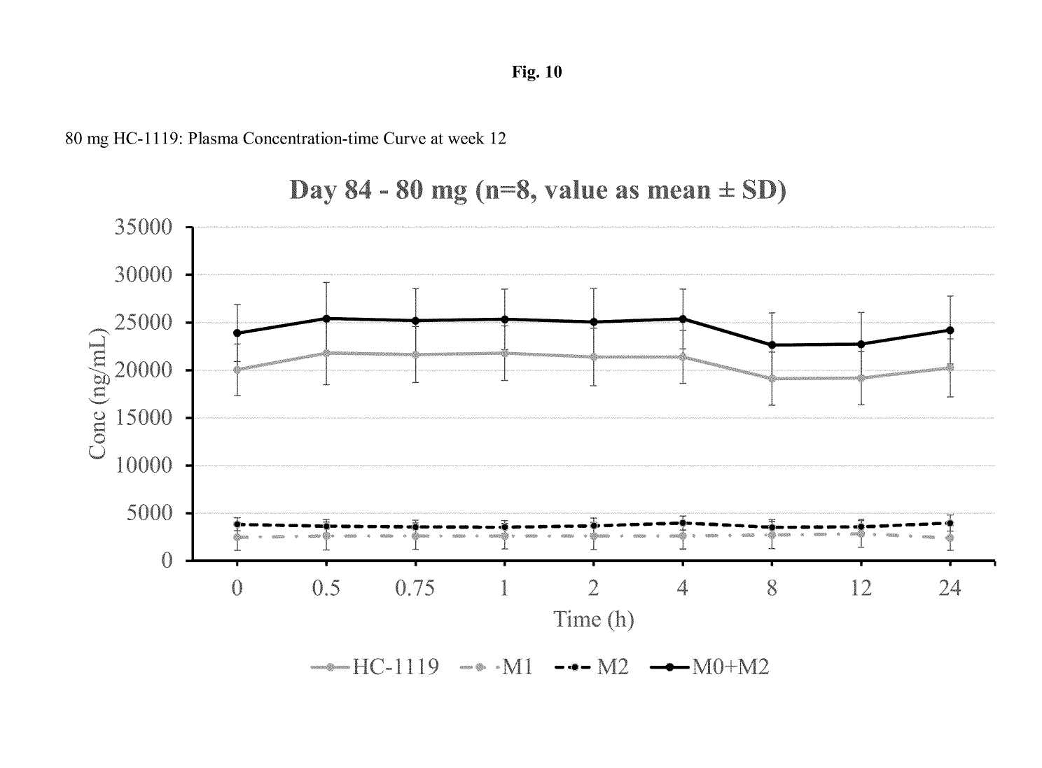

In patients at steady state of 80 mg/day of HC-1119, as provided in FIG. 10, the C_{trough} ratio of M0:M2:M1 is 1:0.18:0.1, showing that the deuteration stabilizes M0 by blocking the generation of M2 and M1. Patients, at steady state 80 mg/day HC-1119 had a PK profile similar to 160 mg/day enzalutamide in terms of Cₘₐₓ, C_{trough}, and AUC0-24 h, showing the higher efficacy of HC-1119.

The following table shows that M2 more readily cross the blood-brain barrier (BBB) in Mice. Table 2 shows the brain penetration of parental drug (M0), M1, and M2 in the PD study in which study, mice (N=10, po, qd) were dosed were with 10 mg/kg of compounds (HC-1119 or enzalutamide) for 28 consecutive days and the parental drug and their major metabolites M1/M2 analyzed from samples collected at 24 h post last dosing.

TABLE 2
Brain penetration of parental drug (M0), M1, and M2

| | | Plasma | Brain/ | Brain |
|---|---|---|---|---|
| | Brain (ng/g) | (ng/ml) | Plasma | penetration |
| HC-1119-M0 HC-1119-M1 HC-1119-M2 | 1393 Not detected 18 | 4686 161 15 | 0.3 Not calculated 1.2 | $\frac{M 2 ( \frac{brain}{plasma} )}{M 0 ( \frac{brain}{plasma} )} = 4$ |
| Enzalutamide-M0 Enzalutamide-M1 Enzalutamide-M2 | 647 Not detected 48 | 2098 199 51 | 0.31 Not calculated 0.94 | $\frac{M 2 ( \frac{brain}{plasma} )}{M 0 ( \frac{brain}{plasma} )} = 3$ |

As shown above, M2 more readily crosses the BBB. Seizures occurred in approximately 1% of patients treated with enzalutamide in clinical trials. It is believed that the brain exposure of (M0+M2) is the cause of seizure inductions. The fact that HC-1119 had a great reduction in M2 both in animals and patients makes HC-1119 have less brain exposure to (M0+M2) than enzalutamide at the same plasma exposure levels. Thus, HC-1119 has a lower potential for inducing epilepsy in patients.

All literature mentioned in the present application are hereby incorporated by reference, as though individually recited herein. Additionally, it should be understood that after reading the above teaching, many variations and modifications may be made by the one of skill in the art, and these equivalents also fall within the scope as defined by the appended claims.

1. A unit dosage form comprising a compound of Formula I in a dosage of about 80 mg, or a crystal form, pharmaceutically acceptable salt, hydrate or solvate thereof, and at least a pharmaceutically acceptable carrier, wherein the compound of Formula I is represented as

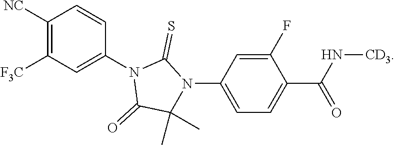 [SMILES: [2H]C([2H])([2H])NC(=O)c1ccc(N2C(=S)N(c3ccc(C#N)c(C(F)(F)F)c3)C(=O)C2(C)C)cc1F]

2. A method of reducing the plasma concentration of prostate specific antigen (PSA) in a subject with elevated PSA by at least 50% comprising administering to said subject the unit dosage form of claim 1.

3. The method of claim 2, wherein the concentration of PSA is reduced by at least 70%.

4. The method of claim 2, wherein the concentration of PSA is reduced by at least 90%.

5. The method of claim 2, further comprising, prior to the step of claim 2, determining the subject as having a PSA concentration of about 5 ng/ml or higher.

6. The method of claim 2, wherein the compound is administered once a day.

7. The method of claim 2, wherein the compound is administered twice a day.

8. The method of claim 2, wherein the compound is administered once every N days, wherein N is an integer between two (2) and eight (8), both inclusive.

9. The method of claim 2, wherein the compound is administered over a period of at least five (5) weeks.

10. The method of claim 2, wherein the compound is administered over a period of at least eight (8) weeks.

11. The method of claim 2, wherein the compound is administered over a period of at least twelve (12) weeks.

12. The method of claim 2, wherein the compound is administered in an oral, parenteral, subcutaneous, intradermal, intramuscular, intravenous, intraarticular, intranasal, intramedullary, intraperitoneal, transmucosal, transdermal, rectal, topical, dermal, buccal, sublingual or intraocular manner.

13. The method of claim 2, wherein the compound is administered orally.

14. The method of claim 2, further comprising determining the subject as currently having, or having been previously diagnosed with prostate cancer.

15. The method of claim 2, further comprising determining the subject as currently having prostate cancer and having never been treated for said cancer.

16. The method of claim 2, wherein the subject was determined as being previously diagnosed with prostate cancer and having undergone previous treatment for prostate cancer.

17. The method of claim 14, wherein the prostate cancer is any wild type androgen receptor (AR) positive prostate cancer and AR mutant positive prostate cancer.

18. The method of claim 14, wherein the prostate cancer is castration resistant prostate cancer (CRPC).

19. The method of claim 14, wherein the prostate cancer is metastatic castration resistant prostate cancer (mCRPC).

20. The method of claim 14, wherein the prostate cancer is non-metastatic castration resistant prostate cancer (nmCRPC).

21. The method of claim 14, furthering comprising determining the prostate cancer as being resistant to the treatment of a single androgen receptor antagonist selected from the group consisting of Flutamide, Nilutamide, Bicalutamide, Abiraterone Acetate, Apalutamide, Darolutamide, Proxalutamide, TRC-253, ONC1-13B, TAK-683, TAK-448, Enzalutamide, SHR3680, GT0918 (Proxalutamide), and ARN509.

22. The method of claim 2, wherein C_{trough} ratio at steady state between the compound of formula I and its metabolite I-M2 ranges from about 5:1 to about 6:1, wherein I-M2 is represented as

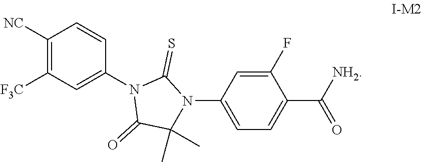 [SMILES: CC1(C)C(=O)N(c2ccc(C#N)c(C(F)(F)F)c2)C(=S)N1c1ccc(C(N)=O)c(F)c1]

23. A method of producing plasma concentrations of a first combination (by weight) of the compound of Formula I and its metabolic product (I-M2) in a subject that is at least 50% more than a second combination (by weight) of enzalutamide and its metabolic product (M2) produced with an identical dosage of enzalutamide by administering at least 40 mg of the compound of Formula I, wherein the I-M2 and the M2 have the same structure as 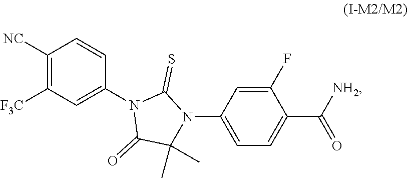 [SMILES: CC1(C)C(=O)N(c2ccc(C#N)c(C(F)(F)F)c2)C(=S)N1c1ccc(C(N)=O)c(F)c1]
and
enzalutamide is represented as

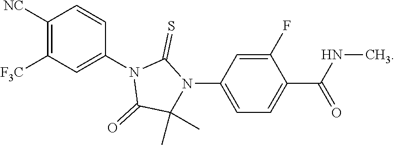 [SMILES: CNC(=O)c1ccc(N2C(=S)N(c3ccc(C#N)c(C(F)(F)F)c3)C(=O)C2(C)C)cc1F]

24. The method of claim 23, wherein the plasma concentrations of the first combination of the compound of Formula I and the I-M2 in the subject is at least about 10 μg/mL at steady state.

25. The method of claim 23, comprising administering at least about 80 mg of the compound of Formula I, wherein the plasma concentrations of the first combination of the compound of Formula I and the I-M2 in the subject is at least about 20 μg/mL at steady state.

26. The method of claim 23, wherein the compound is administered once every day.

27. The method of claim 23, wherein the first combination of the compound of Formula I and its metabolic product (I-M2) in the subject is at least about 80% more than the second combination of enzalutamide and its metabolic product (M2),

28. The method of claim 23, comprising administering at least about 160 mg of the compound of Formula I, wherein the plasma concentrations of a combination of the compound of Formula I and the M2 in the subject is at least about 30 μg/mL at steady state.

29. A method of reducing inter-patient variability in the plasma concentrations or AUC of a nonsteroidal antiandrogen (NSAA) compound comprising administering to a subject in need thereof, a therapeutically effective amount of a deuterated analogue of the NSAA compound, wherein the deuterated analogue is a compound of Formula I.

30. The method of claim 29 wherein inter-patient variability in at least one of Cₘₐₓ, C₂₄ₕ, and C_{trough} is reduced.

31. The method of claim 29, wherein at least 80 mg of the deuterated analogue is administered.

32. The method of claim 31, wherein coefficients of variation (CV %) for Cₘₐₓ, C_{trough}, and AUCₗₐₛₜ are less than about 15%, less than about 16% and less than about 16%, respectively.

33. The method of claim 31, wherein C_{trough} ratio at steady state between the compound of Formula I and I-M2 is about 5.5:1.

34. The method of claim 31, wherein the compound of Formula I is administered over a period of at least five (5) weeks.

35. The method of claim 31, wherein the compound is administered once every N days, wherein N is an integer between two (2) and eight (8), both inclusive.

36. The method of claim 29, wherein the subject currently has, or has been previously diagnosed with prostate cancer.

37. The method of claim 29, wherein the subject has never been treated or has undergone previous treatment for prostate cancer.

38. The method of claim 29, wherein the prostate cancer is CRPC.

39. The method of claim 29, wherein the prostate cancer is mCRPC.

40. The method of claim 29, wherein the prostate cancer is nmCRPC.

41. The method of claim 30, wherein the compound of Formula I is administered over a period of at least five (5) weeks.

42. The method of claim 30, wherein the efficacy of the deuterated analogue is higher than the NSAA compound due to the reduced inter-patient variability.

43. The method of claim 30, wherein the plasma concentration of the deuterated analog is higher than the NSAA compound due to the reduced inter-patient variability.

44. The method of claim 30, wherein the T_{1/2} for the deuterated analog is higher than the NSAA compound due to the reduced inter-patient variability.

45. The method of claim 30, wherein the incidents or severity of adverse effects of deuterated analog is reduced relative to the NSAA compound due to the reduced inter-patient variability.

46. A method of reducing side effect of a NSAA compound comprising administering to a subject in need thereof a unit dosage form of claim 1, wherein a deuterated analog of the NSAA compound is the compound of Formula I.

47. The method of claim 46, wherein the side effect is selected from the group consisting of back pain, joint aches, musculoskeletal pain, diarrhea, hot flashes, peripheral edema, low white blood cell count, headache, upper respiratory tract infection, dizziness, and muscle weakness.
